(19) United States
(12) Patent Application Publication
(10) Pub. No.: US 2024/0148404 A1
(43) Pub. Date: May 9, 2024

(54) APPARATUS AND METHOD FOR SEPTAL PUNCH AND DELIVERY AND MANEUVERING OF THERAPEUTIC DEVICE

(71) Applicant: Protaryx Medical Inc., Baltimore, MD (US)

(72) Inventor: Edwin HLAVKA, Minneapolis, MN (US)

(73) Assignee: Protaryx Medical Inc., Baltimore, MD (US)

(21) Appl. No.: 18/348,553

(22) Filed: Jul. 7, 2023

Related U.S. Application Data
(63) Continuation of application No. PCT/US2022/011623, filed on Jan. 7, 2022.
(60) Provisional application No. 63/136,050, filed on Jan. 11, 2021.

(51) Int. Cl. A61B 17/3205; A61M 29/02; A61B 17/00

(52) U.S. Cl. CPC A61B 17/32053; A61M 29/02; A61B 2017/00243; A61B 2017/00305; A61B 2017/00336; A61B 2017/00367; A61B 2017/00982; A61M 2029/025

(57) ABSTRACT

In some embodiments, an apparatus includes a shaft, and a guide coupled and angularly deflectable relative to the shaft via a guide coupler to be transitioned between a delivery configuration and a deployed configuration in which a distal end of the guide points away from a centerline of the shaft. The apparatus further includes an elongate member removably and slidably disposable within a lumen of the guide and extendable distally relative to the distal end of the guide. The apparatus further includes a puncture member slidably disposable within a lumen of the elongate member and extendable distally relative to a distal end of the elongate member. The guide is configured to have the elongate member and the puncture member removed from the lumen of the guide and to receive in the lumen of the guide a therapeutic device, and to be angularly deflected while holding the therapeutic.

### CROSS REFERENCE TO RELATED APPLICATIONS

This application is a continuation of International Patent Application No. PCT/US2022/011623, filed Jan. 7, 2022, which claims priority to and the benefit of U.S. Provisional Application No. 63/136,050, filed Jan. 11, 2021, each entitled “Apparatus and Method for Septal Punch and Delivery and Maneuvering of Therapeutic Device,” the disclosure of each of which is hereby incorporated by reference in its entirety.

### BACKGROUND

Embodiments are described herein that relate to devices and methods for use in accessing, and enabling delivery of a therapeutic device to, the left side of the heart.

Many diseases and disorders, such as, for example, heart failure, atrial fibrillation, mitral valve disease, and others, specifically impact or are addressable in the left side of the heart. Accordingly, many interventional percutaneous cardiac procedures require access to the left side of the heart, including, for example, electrophysiological procedures, left atrial appendage occlusion procedures, mitral valve repair and replacement procedures, atrial shunt procedures, and many more. In additional to therapeutic interventional procedures, indications for access to the left side of the heart also include diagnostic procedures, including, for example, hemodynamic measurements (e.g., left atrial pressure, trans-mitral pressure gradient, etc.). Minimally-invasive access to the left side of the heart is challenging and not without significant risk.

Some catheter-based procedures access the left side of the heart by puncturing the atrial septum (“AS”) of the heart, which separates the left atrium (“LA”) of the heart from the right atrium (“RA”) of the heart. U.S. Pat. No. 11,045,224, entitled “Apparatus and Method for Septal Punch,” (the '224 Patent) discloses embodiments of devices and methods for performing septal punctures that have several advantages over prior known devices and methods. These advantages include providing a staple platform for manipulation of a deflectable catheter, with a rigid shaft spanning the RA from the inferior vena cava (“IVC”) to the superior vena cava (“SVC”)—this shaft permits axial and rotational adjustment of the deflectable catheter. The deflectable catheter then enables independent adjustment of a deflection angle relative to the rigid shaft to target the fossa ovalis (“fossa”, “FO”, or “F”). The extendable catheter further provides a separate and independent axial extension toward the FO. Thus, the disclosed devices provide a stable and adjustable platform from which the FO can then be punctured, and a guidewire delivered via the devices' integral puncturing element (needle) and the needle's lumen.

The '224 Patent discloses that after the transseptal guidewire is delivered, the device is withdrawn over the guidewire, removed from the patient's body, and discarded. The guidewire can then be used to guide delivery of any suitable therapeutic device (or a delivery sheath for such therapeutic device) to the LA. This delivery step and the balance of the clinical intervention would then be performed using only the chose therapeutic device's capabilities to maneuver into and then within the LA.

It would be clinically advantageous to use the devices disclosed in the '224 Patent to assist delivery of a therapeutic device across the septum and into the LA. It would further be advantageous to then use the disclosed device's advantageous features to assist in maneuvering the therapeutic device within the LA.

### SUMMARY

Devices and methods are described herein for use in minimally-invasively accessing various portions of a patient's anatomy, such as, for example, accessing a LA of a heart through a transseptal puncture, delivering a guidewire, and then to assist in delivering and maneuvering a therapeutic device within the LA. In some embodiments, a method includes inserting a septum penetrating device that includes a shaft having (1) a side catheter guide attached thereto via a guide coupler, and (2) a guide stabilizer/actuator (“GSA”) in a delivery configuration and slidably attached thereto, into an IVC of a heart of a patient and an SVC of the heart such that the GSA is disposed in a RA of the heart. The method further includes applying a distal force to the side catheter guide such that a distal end of the side catheter guide deflects laterally about the guide coupler towards a septum of the heart. The method further includes, with the GSA in its delivery configuration in the right atrium of the heart, actuating the guide stabilizer/actuator to transition the GSA from its delivery configuration to a deployed configuration. After initiating the applying the distal force and with the guide stabilizer/actuator in its deployed configuration, disposing the GSA in contact with the side catheter guide to laterally stabilize the side catheter guide relative to the shaft. The method further includes with the distal end of the side catheter guide laterally deflected about the guide coupler towards the septum and laterally stabilized by the GSA, extending a side catheter that is disposed within the side catheter guide distally from the side catheter guide towards and into contact with the septum. The method further includes, with the distal end of the side catheter in contact with the septum, extending a septum penetrator that is slidably disposed within the side catheter distally from the side catheter such that the septum penetrator pierces the septum. The method further includes advancing a guidewire through the septum penetrator into the LA.

The method can also include withdrawing the side catheter and septum penetrator from the side catheter guide and removing it from the septum penetrating device, then inserting into the side catheter guide a therapeutic device, and delivering the distal end of the therapeutic device into the LA. The septum penetrating device can then be adjusted to assist in the maneuvering of the distal end of the therapeutic device, instead of, or in addition to, the maneuvering capability of the therapeutic device itself. In an alternative embodiment, after the side catheter and septum penetrator have been removed from the septum penetrating device, a transseptal sheath and dilator can be inserted into the side catheter guide and their distal ends delivered to into the LA. The dilator can then be withdrawn from the transseptal sheath and the septum penetrating device, and a therapeutic device can be inserted into the transseptal sheath and its distal end delivered into the LA, and maneuvered as needed.

### BRIEF DESCRIPTION OF THE DRAWINGS

FIG. 1A is a schematic illustration of a septum puncture device, disposed in a delivery configuration, according to an embodiment.

FIG. 1B is a schematic illustration of the septum puncture device of FIG. 1A, disposed in a deployed configuration.

FIG. 2A is a schematic illustration of the septum puncture device of FIG. 1A, disposed in the delivery configuration within a right atrium (“RA”) of a heart of a patient, and coupled to a first guide wire extending from an inferior vena cava (“IVC”) of the heart across the RA and into a superior vena cava (“SVC”) of the heart.

FIG. 2B is a schematic illustration of the septum puncture device of FIG. 1A, disposed in the deployed configuration and such that it has accessed and delivered to the LA a second guide wire.

FIG. 3 is a flowchart illustrating a method of using a septum puncture device to access a left atrium of a heart of a patient, according to an embodiment.

FIG. 4A is a schematic illustration of a septum puncture device, disposed in a delivery configuration, according to an embodiment.

FIG. 4B is a schematic illustration of the septum puncture device of FIG. 4A, disposed in a deployed configuration.

FIG. 5A illustrates a portion of a septum puncture device in side view, disposed in a deployed configuration, according to an embodiment.

FIG. 5B illustrates the septum puncture device of FIG. 5A in side perspective view.

FIG. 5C illustrates a portion of the septum puncture device of FIG. 5A in side view.

FIG. 5D illustrates a portion of the septum puncture device of FIG. 5A in top perspective view.

FIG. 5E illustrates a portion of the septum puncture device of FIG. 5A in bottom perspective view.

FIGS. 6A to 6M are schematic illustrations of a septum puncture device and therapeutic device, according to an embodiment.

FIG. 7A illustrates a portion of a septum puncture device in side view, disposed in a deployed configuration, according to an embodiment, and FIGS. 7B to 7G illustrate a sequence of operation of the septum puncture device of FIG. 7A to deliver a therapeutic device into, and assist in maneuvering the therapeutic device in, the LA, according to an embodiment.

FIG. 8 is a flow chart showing the sequence of operation of the septum puncture device illustrated in FIGS. 7A to 7G.

FIGS. 9A to 9G illustrates a sequence of operation of the septum puncture device of FIG. 7A to deliver a therapeutic device into, and assist in maneuvering the therapeutic device in, the LA, with the additional use of a transseptal sheath and dilator, according to an embodiment.

FIG. 10 is a flow chart showing the sequence of operation of the septum puncture device illustrated in FIGS. 9A to 9G.

### DETAILED DESCRIPTION

Devices and methods are described herein for use in accessing the left side of the heart (e.g., LA) from the right side of the heart (e.g., RA) without requiring open-heart surgery. The methods described herein are minimally invasive and utilize a septum puncture device to access the left side of the heart in a safe (e.g., atraumatic), efficient, timely, accurately and precisely located and repeatable manner. This is accomplished, in part, by providing a steerable (e.g., translatable and rotatable) stable platform between the IVC and SVC from which a puncture member can be extended laterally and into a target puncture location (e.g., the FO) of the atrial septum to deliver a guidewire into the LA, and then to enable delivery of a therapeutic device into the LA and assist in maneuvering the therapeutic device. As discussed above, a therapeutic device can be any device that may be desirable to deliver into the left atrium for therapeutic procedures therein (or in other parts of the anatomy accessible via the left atrium), including suitable devices for electrophysiological procedures (e.g. ablation), left atrial appendage occlusion procedures, mitral valve repair and replacement procedures, atrial shunt procedures, etc. Other devices that may be desirable to deliver into the left atrium (and beyond) can be any device suitable for diagnostic procedures, including, for example, hemodynamic measurements (e.g., left atrial pressure, trans-mitral pressure gradient, etc.), electrophysiological mapping, etc. and/or for imaging procedures (intracardiac echocardiography (ICE), intravascular ultrasound (IVUS), etc.), etc. For purposes of this disclosure, a “therapeutic device” can include any device usable for therapeutic, diagnostic, imaging, or other procedures, including all those described above.

In some embodiments, a method includes inserting a shaft having (1) a side catheter guide attached thereto via a guide coupler, and (2) a guide stabilizer/actuator (“GSA”) in a delivery configuration and slidably attached thereto, into an inferior vena cava of a heart of a patient and a superior vena cava of the heart such that the guide stabilizer/actuator is disposed in a right atrium of the heart. The method further includes applying a distal force to the side catheter guide such that a distal end of the side catheter guide deflects laterally about the guide coupler towards a septum of the heart. The method further includes, with the guide stabilizer/actuator in its delivery configuration in the right atrium of the heart, actuating the guide stabilizer/actuator to transition the guide stabilizer/actuator from its delivery configuration to a deployed configuration. After initiating the applying the distal force and with the guide stabilizer/actuator in its deployed configuration, disposing the side catheter guide in contact with the side catheter guide to laterally stabilize the side catheter guide relative to the shaft. The method further includes with the distal end of the side catheter guide laterally deflected about the guide coupler towards the septum and laterally stabilized by the guide stabilizer/actuator, extending a side catheter that is disposed within the side catheter guide distally from the side catheter guide towards and into contact with the septum. The method further includes, with the distal end of the side catheter in contact with the septum, extending a septum penetrator that is slidably disposed within the side catheter distally from the side catheter such that the septum penetrator pierces the septum. The method further includes advancing a guidewire through the septum penetrator into the LA.

The method can also include withdrawing the side catheter and septum penetrator from the side catheter guide and removing it from the septum penetrating device, then inserting into the side catheter guide a therapeutic device, and delivering the distal end of the therapeutic device into the LA. The septum penetrating device can then be adjusted to assist in the maneuvering of the distal end of the therapeutic device, instead of or in addition to the maneuvering capability of the therapeutic device itself. In an alternative embodiment, after the side catheter and septum penetrator have been removed from the septum penetrating device, a transseptal sheath and dilator can be inserted into the side catheter guide and their distal ends delivered to into the LA. The dilator can then be withdrawn from the dilator and the septum penetrating device, and a therapeutic device can be inserted into the transseptal sheath and its distal end delivered into the LA, and maneuvered as needed.

As used herein, the terms “proximal” and “distal” refer to the direction closer to and away from, respectively, an operator (e.g., a surgeon, physician, nurse, technician, etc.) who would insert the septum puncture device into the patient, with the tip-end (i.e., distal end) of the device inserted inside a patient's body first. Thus, for example, the end of a main shaft described herein first inserted inside the patient's body would be the distal end, while the opposite end of the main shaft (e.g., the end of the main shaft being manipulated by the operator) would be the proximal end of the main shaft.

As used herein, the terms “advance,” “advanced,” and “advancing” each refer to distal movement. Advancing a device within a patient's vasculature, for example, refers to moving at least a portion of the device distally within the patient's vasculature. Similarly, as used herein, the terms “withdraw,” “withdrawn,”, and withdrawing” each refer to proximal movement. Withdrawing a device within a patient's vasculature, for example, refers to moving at least a portion of the device proximally within the patient's vasculature. In some instances, advancing and withdrawing can refer to relative movement of the device itself. Advancing a side catheter, for example, can refer to moving a side catheter distally relative to a side catheter guide to which the side catheter is movably coupled. Similarly, withdrawing the side catheter, for example, can refer to moving the side catheter proximally relative to the side catheter guide to which the side catheter is movably coupled.

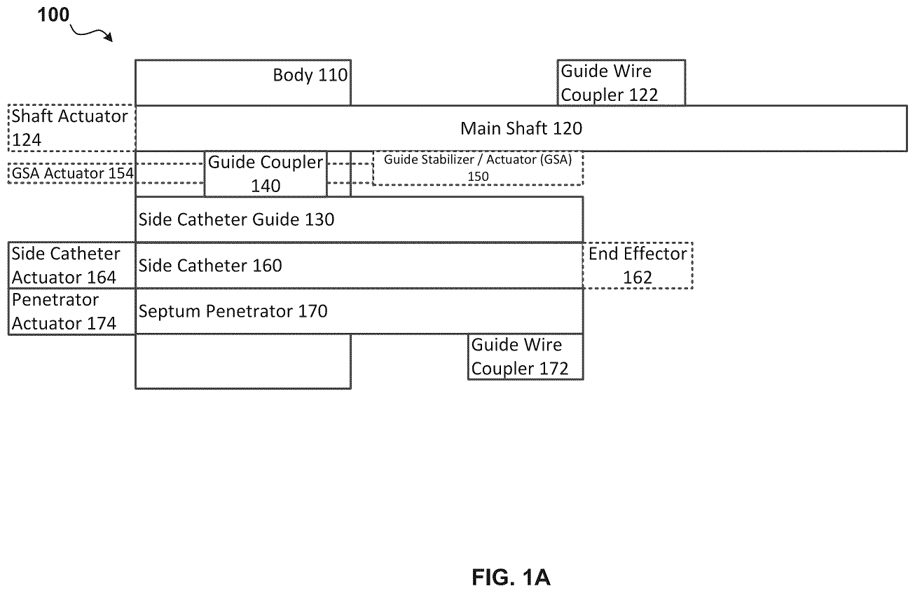

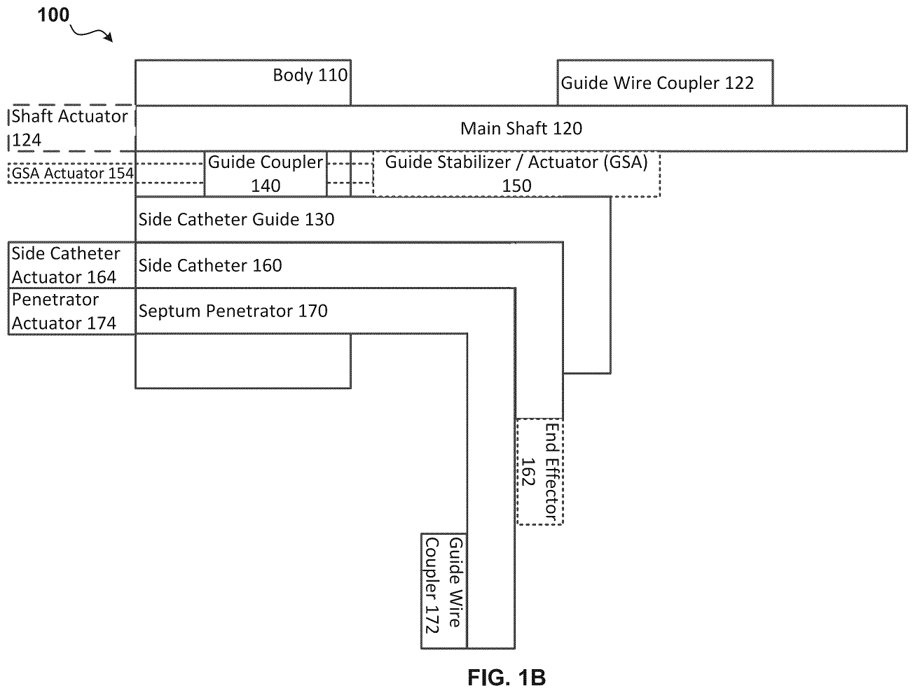

The septum puncture device 100 can be used to access a left side of the heart (e.g., left atrium) from the right side of the heart (e.g., right atrium), to deliver a guidewire to the left side of the heart, and then to assist in delivery of a therapeutic device to the left atrium, and maneuvering of the therapeutic device within the left atrium. As shown in FIG. 1, the septum puncture device 100 includes a body 110 coupled to a main shaft 120, a side catheter guide 130, a side catheter 160, and a septum penetrator 170. The main shaft 120 is coupled to the side catheter guide 130 via a guide coupler 140, the side catheter guide 130 is coupled to the side catheter 160, and the side catheter 160 is coupled to the septum penetrator 170, as shown in FIG. 1A. The side catheter guide 130 is configured to define a pathway through or across which the side catheter 160 can travel (e.g., be advanced and/or withdrawn). Said another way, and as described in further detail herein, the side catheter guide 130 can be manipulated (e.g., actuated from a delivery state to a deployed state) to guide the side catheter 160 in a desired direction (the actuated or deployed state of the side catheter guide 130 is shown in FIG. 1B), e.g., towards the left atrium.

As described in further detail herein, the guide coupler 140 can couple the side catheter guide 130 to the main shaft 120 to minimize or prevent relative translational movement between the main shaft 120 and the side catheter guide 130, but to allow relative rotational movement between the main shaft 120 and the side catheter guide 130, as illustrated schematically in FIG. 1B. In this manner, the guide coupler 140 can facilitate transition of the side catheter guide 130 from a delivery configuration (e.g., parallel to or substantially parallel to the main shaft 120), e.g., for insertion through the patient's vasculature and into the RA, to a deployed configuration such that a distal end of the side catheter guide 130 is deflected laterally (e.g., perpendicular or substantially perpendicular) relative to the main shaft 120, e.g., towards the patient's left atrium (e.g., the FO of the atrial septum). In some embodiments, the guide coupler 140 can be a hinge to facilitate lateral deflection of the side catheter guide 130 relative to the main shaft 120, as described in further detail herein. In such embodiments, for example, a distal force can be applied to a proximal end portion of the side catheter guide 130, thereby causing the hinge to rotate and cause a distal end portion of the side catheter guide (i.e., a portion of the side catheter guide 130 that extends distal to the guide coupler 140) to laterally deflect. In some implementations, the amount of lateral deflection or the defined between the side catheter guide 130 and the main shaft 120 after such lateral deflection is adjustable by the operator intra-procedure, i.e., in real-time, such that, for example, the operator has procedural flexibility when locating the target puncture location.

In some implementations, one or more of the main shaft 120, the side catheter guide 130, or the side catheter 160 can have a circular cross-sectional shape, while in other implementations, one or more of the main shaft 120, the side catheter guide 130, or the side catheter 160 can have a non-circular cross-sectional shape. In some instances, for example, the main shaft 120 and the side catheter guide 130 can have circular cross-sectional shapes, and can be operably coupled together, as discussed in further detail herein, such that the main shaft 120 and the side catheter guide 130 are at least partially disposed side-by-side (e.g., during delivery). In other instances, for example, the main shaft 120 may have a non-circular cross-section (e.g., a half-moon shape, c-shape a convex or concave shape, or any other suitable noncircular cross-sectional shape) such that when coupled to the side catheter guide 130, a portion of the side catheter guide 130 can be nestled within a space defined at least in part by the non-circular curvature of the main shaft 120. In this manner, the collective cross-sectional area, footprint, diameter, etc. of the main shaft 120 and side catheter guide 130 can be reduced. In some instances, a similar relationship can be had by the main shaft 120 and the side catheter 160 (e.g., in embodiments in which a septum puncture device does not have a side catheter guide).

In some embodiments, the septum puncture device 100 includes a side catheter guide stabilizer/actuator (“GSA”) 150 (also referred to herein as “guide stabilizer/actuator”), and a GSA actuator 154 operably coupled to the GSA 150 and configured to actuate the GSA 150. In some implementations, the GSA 150 can be configured to stabilize (e.g., laterally, axially (proximally or distally), e.g., with respect to the main shaft 120) the side catheter guide 130 to facilitate the side catheter's 160 engagement with the FO and the septum penetrator's 170 penetration of the FO. In this manner, the guide coupler 140 can laterally deflect the side catheter guide 130, and the GSA 150 can stabilize the side catheter guide 130 (and in turn the side catheter 160, optional end effector 162, and septum penetrator 170) to optimize subsequent penetration of the septum and access to the left atrium. In some implementations, in addition to or instead of stabilizing the side catheter guide 130, the GSA 150 can be configured to laterally deflect (e.g., laterally deflect in addition to the lateral deflection caused or facilitated by the guide coupler 140, as described above) the side catheter guide 130 (and in turn the side catheter 160 and septum penetrator 170, given their coupling to the side catheter guide 130). In this manner, in some implementations, the guide coupler 140 and the GSA 150 can collectively laterally deflect and stabilize the side catheter guide 130 (and in turn the side catheter 160, optional end effector 162, and septum penetrator 170) to optimize subsequent penetration of the septum and access to the left atrium.

The GSA 150 can be manipulatable in any manner suitable to provide the above-described functionality. In some embodiments, for example, the GSA 150 can be a balloon, and as such, it can be configured to be inflatable and deflatable. In such embodiments, the GSA 150 can be fluidically coupled to a lumen extending from the GSA 150 to the GA actuator 154 such that the GA actuator 154 can selectively deliver fluid to the GA actuator 154 to inflate the GSA 150 (i.e., deploy the GSA 150), and selectively withdraw fluid from the GSA 150 to deflate the GSA 150 for removal of the GSA 150 from the heart (e.g., after left atrium access has been achieved).

In embodiments in which the GSA 150 is a balloon, the balloon can have any shape and size suitable to perform the desired functions described herein. In some embodiments, for example, the balloon can be cone-shaped, while in other embodiments, it can be at least partially concave, convex, circular, oval, or the like. Further, in some embodiments, the balloon can have one or more lobes, e.g., it can be bi-lobed or tri-lobed, to, for example, allow blood flow along the balloon and past the device. Further, the balloon can have additional features configured to improve stabilization of the side catheter guide 130 (e.g., improve coupling between the balloon and the side catheter guide 130). In some embodiments, for example, a balloon can have dimples, protrusions, ridges, adhesives, etc.

The balloon can be formed of any material or combination of materials suitable to perform its functionality described herein. In some embodiments, for example, the balloon can be formed of one or more of Polyethylene, Polyethylene terephthalate (“PET”), a polymer, a thermoplastic polymer, an elastomer, nylon, polyurethane, any non-compliant material, etc. The balloon can be configured to be inflated to any suitable pressure, e.g., from about 2 ATM to about 20 ATM, as an example. In some instances, higher inflation pressures can result in greater or improved rigidity of the balloon, thereby providing better stabilization of the side catheter guide, side catheter, septum penetrator, etc.

The GSA 150 can be formed of any material suitable to perform its functions described herein. In some embodiments the GSA 150 can include or be formed of shape memory material (e.g., Nitinol) and configured to be transitioned between a delivery/withdrawal configuration in which the GSA 150 is constrained, compressed, or otherwise placed in a relatively small arrangement, and a deployed configuration in which the GSA 150 is unconstrained, expanded, or otherwise placed in a larger arrangement sufficient to laterally deflect or stabilize the side catheter guide 130 as described in further detailed herein.

Similar to the guide coupler 140, in some embodiments, the GSA 150 can include or be formed of radiopaque material to assist the operator in locating that portion of the septum puncture device 100 before, during, or after deployment. In this manner, the operator can in real time selectively position the septum penetrator 170 in a position suitable to penetrate the FO upon actuation of the septum penetrator 170. In embodiments in which the GSA 150 is a balloon, for example, in some instances the GSA 150 can be inflated with a contrast agent (or a combination of a contrast agent and another fluid, such as saline) to provide visualization (e.g., under any suitable imaging modality) for the operator when the GSA 150 is disposed within the patient.

As described in further detail herein, with the side catheter guide 130 laterally deflected and stabilized at a suitable angle relative to the FO or the main shaft 120, and with (1) one or more landmark portions of the septum puncture device 100 and (2) a desired puncture location (e.g., the FO) on the septum visible to the operator from outside the patient, the operator can manipulate the main shaft 120 translationally or rotationally in any suitable manner to align the side catheter guide 130 with the FO.

Further as shown in FIG. 1A, the septum puncture device 100 includes a guide wire coupler 122 configured to couple the main shaft 120 to a guide wire (not shown in FIG. 1A) to facilitate delivery of the septum puncture device 100 into a patient (e.g., through the vasculature of the patient) and to the patient's heart, and a guide wire coupler 172 configured to couple a guide wire (not shown in FIG. 1A) to the septum penetrator 170, to facilitate delivery of that guide wire to the left side of the heart (e.g., the left atrium).

Further as shown in FIG. 1A, the septum puncture device 100 optionally includes a shaft actuator 124 operably coupled to the main shaft 120 and configured to actuate the main shaft 120 to advance or withdraw the main shaft 120 relative to the body 110. The septum puncture device 100 further includes (1) a side catheter actuator 164 operably coupled to and configured to actuate the side catheter 160 to advance or withdraw the side catheter 160, thereby transitioning the side catheter 160 between a delivery configuration and a deployed configuration (the side catheter 160 shown in an actuated or deployed configuration in FIG. 1B), and a (2) a septum penetrator actuator (or “penetrator actuator”) 174 to actuate the septum penetrator 170 to advance or withdraw the septum penetrator 170, thereby transitioning the septum penetrator 170 between a delivery configuration and a deployed configuration (the septum penetrator 170 shown in an actuated or deployed configuration in FIG. 1B), as described in further detail herein.

Further as shown in FIG. 1A, the septum puncture device 100 optionally includes a GSA (“GA”) 150 coupled to the main shaft 120. The optional GSA 150 is operably coupled to a GA actuator 154 that is configured to actuate the GSA 150, as described in further detail herein.

Further as shown in FIG. 1A, the septum puncture device 100 optionally includes an end effector 162 coupled to and extending distally from the side catheter 160. The end effector 162 is configured to facilitate subsequent puncture through a target puncture location, such as, for example, the FO of the septum of the heart. The end effector 162 can be configured, for example, to contact or tent the FO, as described in further detail herein. Such contact or tenting of the FO can, for example, reduce or minimize the force required to penetrate the FO and/or provide for improved force distribution to the FO. The end effector 162 can be configured to prevent inadvertent puncturing of and/or damage to the FO with the end effector 162.

In some embodiments, the end effector 162 is formed of or includes a radiopaque material such that the end effector 162 can be visualized when within the heart from outside the patient under any suitable imaging modality (e.g., fluoroscopy, echocardiography, etc.), to facilitate an operator in deploying the end effector 162, e.g., locating the end effector 162 within the heart or relative to the FO in preparation for deploying the septum penetrator 170.

In some embodiments, the end effector 162 can include multiple configurations, e.g., a delivery or withdrawal configuration, in which the end effector 162 is configured to be routed through the patient's vasculature, and a deployed configuration in which the end effector 162 is configured to facilitate subsequent penetration of the FO, as described in further detail herein. In such embodiments, for example, the end effector 162 can be delivered to the heart in a compressed, deflated, or otherwise relatively small configuration, and then transitioned into a deployed configuration in which it is expanded, inflated, or otherwise increased in size to then contact or tent the FO. Further, in some embodiments, after deployment of the end effector 162, the end effector 162 can be transitioned to a withdrawal configuration (which can be the same as or similar to its delivery configuration) in which the end effector 162 is in a compressed, deflated, or otherwise small configuration to assist in removal of the end effector 162 from the patient.

The end effector 162 can be formed of any suitable material(s) to facilitate its functionality described herein. In some embodiments, for example, the end effector 162 can be formed of shape memory material(s) (e.g., Nitinol) or a polymer, or a combination thereof (e.g., Nitinol coated with a polymer), such that it can be transitioned between a constrained or compressed arrangement (e.g., delivery or withdrawal configuration) and an unconstrained or expanded arrangement (deployed configuration). In some embodiments, for example, the end effector 152 can be or include a balloon such that it can be delivered to the heart in a deflated arrangement and then inflated (e.g., via an inflation lumen fluidically coupled to and extending proximally from the end effector 162, not shown) to a deployed configuration. Various further embodiments of an end effector are described in further detail below.

Each of the main shaft 120, the guide wire coupler 122, the side catheter guide 130, the guide coupler 140, the optional GSA 150, the side catheter 160, the septum penetrator 170, and the guide wire coupler 172 are translatable (e.g., distally advanceable and/or extendable, and proximally withdrawable and/or retractable) relative to the body 110. The side catheter 160 is translatable relative to the side catheter guide 130, and the septum penetrator 170 is translatable relative to the side catheter 160, as described in further detail herein.

The septum penetrator 170 can be sized, shaped, and formed of any material suitable to effectively penetrate and traverse a target tissue such as the FO. In some embodiments, for example, the septum penetrator 170 can be a needle. In some embodiments, the septum penetrator 170 can be a non-coring needle (e.g., a needle with a sharp tip that has a cutting edge, such as, for example, a Quincke-type needle). In some embodiments, the septum penetrator 170 can have variable material properties. In such embodiments, for example, a distal portion of the septum penetrator 170 can have a stiffness greater than a stiffness of a portion proximal to that distal portion. In this manner, the stiffer distal portion can be configured for penetration through the septum, while the portion proximal can be configured for delivery through the patient's vasculature. In some embodiments, the septum penetrator 170 can be solid-tipped and can be electrified with radiofrequency (“RF”) energy to puncture the FO.

The septum penetrator 170 can have any suitable length, for example, any length suitable to reach the LA. In some embodiments, for example, the septum penetrator 170 can have an effective length (i.e., the length extendable from the distal end of the side catheter 160 (or from the distal end of the end effector 162) of about 5 mm to about 25 mm. In some instances, an effective length of the septum penetrator 170 can be about 8 mm or about 10 mm, or any length therebetween. In some embodiments, the septum penetrator 170 can contain or be configured to receive a stylet to limit or minimize tissue coring. In some embodiments, the septum penetrator 170 can include a pressure transducer (not shown) configured to monitor pressure through a lumen of the septum penetrator 170. In some embodiments, a port or Luer lock can be incorporated into the septum puncture device 100 to flush the septum penetrator 170.

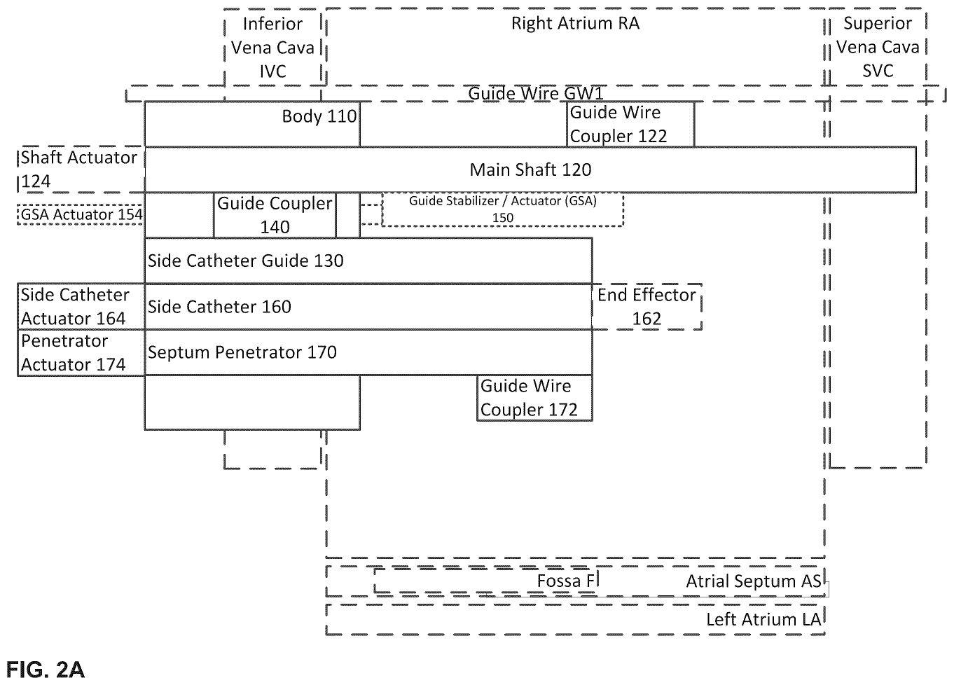

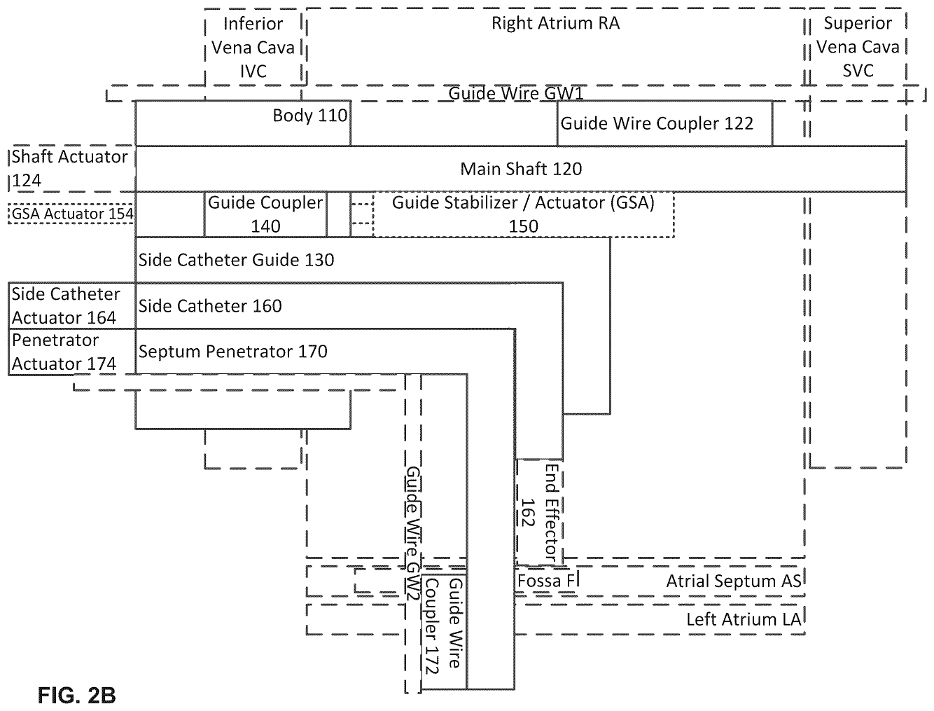

Turning to FIGS. 2A and 2B to describe the septum puncture device 100 (1) in context with the anatomy of a patient and (2) in a sample procedure to access the LA of the patient, FIG. 2A is a schematic illustration of the septum puncture device 100 disposed in a delivery configuration within the RA of the heart and coupled to a first guide wire GW1 extending from the IVC across the RA and into a SVC and FIG. 2B is a schematic illustration of the septum puncture device 100 disposed in a deployed configuration and such that it has accessed and delivered to the LA a second guide wire that can be used to provide subsequent access to the LA.

In use, prior to introducing into the patient the septum puncture device 100, a guide wire GW1 can be inserted through an entry site of the patient (e.g., femoral vein puncture site) (not shown) and advanced through the patient's vasculature across the IVC and RA, and into the SVC using known, suitable techniques for guidewire delivery. With the guide wire GW1 disposed in such a manner, the septum puncture device 100 can be movably coupled to the guide wire GW1 via the guide wire coupler 122 and advanced from the entry site of the patient towards the heart. In some embodiments, the guide wire coupler 122 can be a lumen defined by the main shaft 120 through which the guide wire GW1 can be disposed and such that the main shaft 120 can be slidably disposed about the guide wire GW1. The guide wire GW1 can be any suitable size. In some embodiments, for example, the guide wire GW1 can have a diameter of about 0.014 inches to about 0.035 inches in diameter. In some embodiments, the guide wire GW1 can be about 0.025 inches diameter. With the guide wire coupler 122 movably coupled to the delivered guide wire GW1, the septum puncture device 100 can be advanced along the guide wire GW1 into the heart, as shown in FIG. 2A. More specifically, with the main shaft 120 coupled to (1) the body 110 and (2) the side catheter guide 130 via the guide coupler 140, the body 110, the main shaft 120, the guide coupler 140, the side catheter guide 130, the side catheter 160, the septum penetrator 170, and the guide wire coupler 172 all can be advanced into the heart of the patient as shown in FIG. 2A, such that body 110 extends through the IVC and into the RA, and the main shaft 120 extends into the SVC. With the main shaft 120 spanning the IVC, RA, and SVC, the main shaft 120 can provide a foundation or backstop against which the side catheter guide 130, side catheter 160, and septum penetrator 170 can be deployed and advanced towards the septum, as described in further detail herein.

In some instances, a distal end of the (1) main shaft 120, (2) side catheter guide 130, (3) side catheter 160, and septum penetrator 170 (and accompanying couplers, e.g., the guide wire coupler 122 and the guide wire coupler 172), can be disposed within the body 110 (e.g., within one or more lumens (not shown) defined by the body 110). In this manner, during delivery, the patient's anatomy can be protected or shielded by the body 110 to avoid inadvertent trauma to or contact with the patient's anatomy from such components. With a distal end of the body 110 disposed in or near the RA, the body 110 can be withdrawn (and/or one or more of the components movably coupled thereto can be advanced), thereby exposing the side catheter guide 130 and guide coupler 140 within the RA.

With the side catheter guide 130 exposed within the RA and translationally fixedly coupled to the main shaft 120 via the guide coupler 140, the side catheter guide 130 can be actuated to laterally deflect the distal end of the side catheter guide 130 (and as a result, also the side catheter 160, the septum penetrator 170, and the guide wire GW2 if disposed in the side catheter guide 130 during its lateral deflection), as shown in FIG. 2B. The side catheter guide 130 can be laterally deflected at any angle suitable to direct the side catheter 160 and septum penetrator 170, which are movably attached to the side catheter guide 130, towards the target penetration site, e.g., the FO, as shown in FIG. 2B. In some instances, an optimal angle of entry to the FO is 90 degrees or substantially 90 degrees relative to a surface line tangent to the FO, which can be about a similar angle relative to a central axis of the main shaft 120. Such a perpendicular (or substantially perpendicular) angle of entry can minimize the force required to penetrate the FO because the entire or substantially entire force vector is directed at the plane of the FO (rather than a tangential approach). Additionally, such a perpendicular (or substantially perpendicular) angle of entry, given the nature of a patient's anatomy, directs the septum penetrator 170 to a relatively large open space within the LA, thereby minimizing risk of inadvertent puncture within the LA (e.g., inadvertent puncture of a wall of the LA).

In other instances, the angle of entry relative to the FO or relative to the central axis of the main shaft 120 can be anywhere within a range of about 50 degrees to about 90 degrees. In some instances, the preferred angle of entry can be selected based on a particular therapy planned for the left side of the heart. The angle of entry, for example, defines the trajectory for the subsequent therapeutic device to enter the left side of the heart, and so in some instances an optimal angle and location of entry through the FO is based on a particular therapeutic device or procedure.

Note that the guide wire GW2 can be delivered in any suitable manner. In some instances, for example, the guide wire GW2 is disposed within the side catheter guide 130 during delivery of the side catheter guide 130, while in other instances the guide wire GW2 is inserted at a later time during the procedure, e.g., after the septum penetrator 170 has penetrated the FO and reached the LA.

With the side catheter guide 130 transitioned to its deployed configuration, in which the side catheter guide 130 is laterally deflected towards the FO, the side catheter actuator 164 can be actuated to advance the side catheter 160 along a path defined at least in part by the side catheter guide 130 and towards the FO. In some instances the side catheter 160 is advanced until it's distal end tents or otherwise contacts the FO. For embodiments that include the end effector 162, the side catheter 160 can be advanced until the end effector 162 extending from the distal end of the side catheter 160 tents or otherwise contacts the FO.

In embodiments in which the end effector 162 is expandable and compressible, the end effector 162 can be delivered to the Right Atrium RA in a compressed or relatively small configuration, and then transitioned to a deployed configuration in which the end effector 162 is expanded to a relatively larger configuration, and then advanced to engage with the FO. After sufficient penetration of the Atrial Septum AS with the septum penetrator 170, as described in further detail herein, the end effector 162 can be transitioned to its retracted or compressed configuration suitable to be withdrawn from the patient. In embodiments in which the side catheter 160 is slidably disposed within a lumen defined by the side catheter guide 130, the end effector 162 can similarly be slidably disposed within the lumen defined by the side catheter guide 130 such that the side catheter guide 130 contains the end effector 162 in its constrained or compressed configuration during delivery, and then as the side catheter actuator 164 is actuated to advance the side catheter 160 distally from the distal end of the side catheter guide 130, the end effector 162 can transition to its expanded or unconstrained configuration as or after it exits the lumen of the side catheter guide 130.

With the side catheter 160 (or end effector 162) in sufficient contact with the FO, the penetrator actuator 174 can be actuated to advance the septum penetrator 170 relative to and along a path defined at least in part by the side catheter 160. The septum penetrator 170 can be advanced through the FO and across the Atrial Septum AS and into the Left Atrium LA. In some embodiments, the side catheter 160 defines a lumen through which the septum penetrator 170 is slidably disposed such that actuating the penetrator actuator 174 advances the septum penetrator 170 through the lumen of the side catheter 160. The septum penetrator 170 can be advanced in this manner to penetrate the FO and to extend into the left atrium LA. During such penetration, the main shaft 120 can provide lateral or axial stability to the septum penetrator 170.

As the distal end of the septum penetrator 170 is advanced across the Atrial Septum AS and into the Left Atrium LA, the guide wire GW2 can follow via the guide wire coupler 172 and the septum penetrator 170 in instances in which the guide wire GW2 is coupled to the side catheter guide 130 during delivery of the side catheter guide 130. In other instances, the guide wire GW2 can be inserted at a later time during the procedure, e.g., after the septum penetrator 170 has penetrated the FO and reached the LA In some embodiments, the guide wire coupler 172 is a lumen defined by the septum penetrator 170 and through which the guide wire GW2 can be slidable disposed. In such embodiments, the guide wire GW2 can be disposed within the lumen of the septum penetrator 170 during delivery and deployment of the septum penetrator 170 into the Left Atrium LA.

With the septum penetrator 170 and the guide wire GW2 disposed within the Left Atrium LA, the guide wire GW2 can be further advanced into the Left Atrium LA by manipulation of the guide wire GW2 at its proximal end, and/or the septum penetrator 170 can be withdrawn from the Left Atrium LA, across the puncture or entry site of the FO, leaving the guide wire GW2 within the Left Atrium LA.

With the guide wire GW2 delivered to the Left Atrium LA, and extending proximally from the Left Atrium LA across the puncture or entry site of the FO, into the Right Atrium RA, the IVC, and through the vasculature of the patient to the entry point of the patient (for subsequent access to the Left Atrium AS), the septum puncture device 100 can be withdrawn from the heart proximally over guide wire GW2 and from the patient.

The guide wire GW2 can be any guide wire suitable to provide desirable subsequent access to the Left Atrium LA. In some embodiments, for example, the guide wire GW2 can be a pigtail, atraumatic guide wire or other suitable guide wire conventionally used in transseptal procedures. For example, the guide wire GW2 can have a flexible, spiral tip, pigtail, and can be configured to anchor the septum puncture device 100 to the LA, thereby limiting or preventing the guide wire GW2 from being inadvertently withdrawn or removed from the LA in response to or while the septum puncture device 100 is being withdrawn along the guide wire GW2 and from the patient. Another example guide GW2 can be a ProTrack™ Pigtail Wire from Baylis Medical Company, Inc.

The septum puncture device 100 can be configured to be withdrawn from the patient in any suitable sequence (e.g., after the guide wire GW2 has been delivered to the Left Atrium LA). With the guide wire GW2 disposed within the Left Atrium LA, for example, the portions of the septum penetrator 170 and guide wire coupler 172 disposed within the Left Atrium LA can be withdrawn relative to the guide wire GW2 and through the puncture site in the FO and into the Right Atrium RA. In embodiments in which the side catheter 160 defines a lumen through which the septum penetrator is slidably disposed, the septum penetrator 170 can be withdrawn relative to and into the lumen defined by the side catheter 160. In this manner, the septum penetrator 170, and particular it's distal that is designed to penetrate tissue, can be sheathed or shielded by the side catheter 160 to facilitate safe withdrawal from the patient and avoid inadvertent contact with the patient's heart or vasculature during removal of the septum puncture device 100 from the patient.

Similarly, the side catheter 160 can be withdrawn relative to the side catheter guide 130. For example, in embodiments in which the side catheter guide 130 defines a lumen through which the side catheter 160 is slidably disposed, the side catheter 160 can be withdrawn into the lumen of the side catheter guide 130. In embodiments in which the septum puncture device 100 includes an end effector 162, the side catheter guide 160 can be withdrawn relative to and into the lumen of the side catheter guide 130 such that the end effector 162 is also withdrawn into the lumen of the side catheter guide 130. In embodiments in which the end effector 162 has a deployed configuration with a diameter larger than an internal diameter of the side catheter guide 130, the end effector 162 can be configured to be transitioned from its deployed configuration to its withdrawal (or delivery) configuration. For example, if the end effector 162 is a balloon, it can be deflated and then withdrawn into the lumen of the side catheter guide 130. As another example, if the end effector 162 includes or is formed of shape memory material, the end effector 162 can be compressed, constrained, or otherwise transitioned to a smaller arrangement such that it can be withdrawn into the side catheter guide 130. In some instances, withdrawal of the end effector 162 into the side catheter guide 130 can cause the end effector 162 to transition to its constrained or compressed configuration.

Further, the side catheter guide 130 can be configured to transition from its deployed configuration in which its distal portion is laterally deflected relative to the main shaft 120 to its withdrawal (or delivery) configuration in which the side catheter guide 130 is at least substantially linear and parallel to the main shaft 120. In some embodiments, for example, a proximal force can be applied to a proximal end portion of the side catheter guide 130 to withdraw the side catheter guide 130 relative to the main shaft.

With the septum puncture device 100 disposed as shown in FIG. 2A, for example, after delivering the guide wire GW2, the septum puncture device 100 can be withdrawn from the heart and from the patient. For example, the body 110, and all of the components coupled thereto, can be withdrawn from the heart, through the patient's vasculature, and out through the initial entry site into the patient (e.g., the femoral puncture site).

Although embodiments described herein refer to introducing a guide wire and septum puncture device into the patient's vasculature, and across the IVC and RA, and into the SVC, access to the RA for purposes of deploying a septum penetrator, can be accomplish in a variety of ways. In some embodiments, for example, the guide wire and septum puncture device can be inserted into a patient's jugular vein (e.g., right internal jugular vein), and then advanced into and across the SVC and RA, and into the IVC, such that a distal end of the septum puncture device is disposed in the IVC (or beyond).

Although embodiments described herein refer to a single FO puncture to deliver a single guide wire to the LA, it should be understood that the septum puncture devices described herein can be used to perform multiple punctures and to deliver multiple guide wires. In some instances, for example, a double puncture and delivery of two guide wires may be desirable, e.g., in connection with an atrial fibrillation ablation procedure. In such instances, the septum puncture devices described herein can be deployed twice to puncture the septum twice, with each puncture providing access to deliver a guide wire, as described herein. In some procedures that require multiple punctures and guide wires delivered to the LA, for example, it can be crucial that the punctures are in a particular location and located a particular distance from each other, and as described through this disclosure, the septum puncture devices described herein provide just that.

Further, instead of using a septum puncture device described herein to administer multiple punctures in series (e.g., with a single penetrator, single side catheter, single side catheter guide, etc.), in some embodiments, any of the septum puncture devices described herein can be modified to incorporate additional components. For example, in some instances, a septum puncture device can include a body and a main shaft (similar to septum puncture device 100), but also include two side catheter guides, two side catheters, two end effectors, two septum penetrators, and two guide couplers (for the guide wires being delivered), and optionally one or two guide couplers and one or two guide stabilizer/actuators. In this manner, two side catheter guides can be deployed (i.e., laterally deflected and stabilized) simultaneously, and then two side catheters (optionally with end effectors) can be advanced, optionally simultaneously, to contact the septum, and then two septum penetrators can be advanced, optionally simultaneously, to penetrate the septum. With two punctures in the septum, two guide wires can then be delivered, optionally simultaneously. In such instances, the preferred distance between the two punctures can be selectively defined by the distance between the side catheters from which the septum penetrators are advanced.

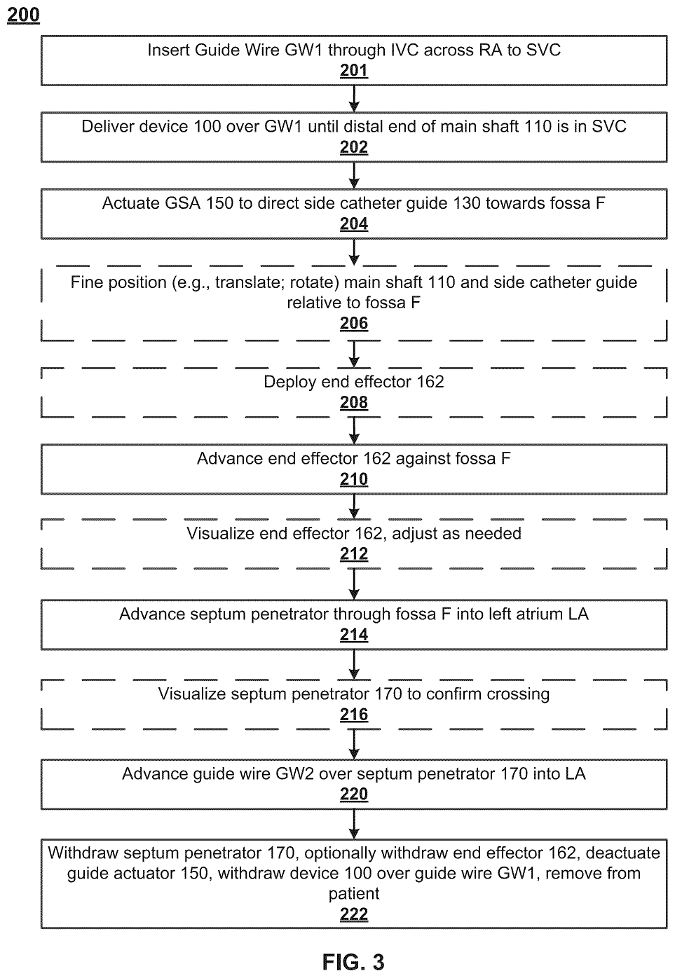

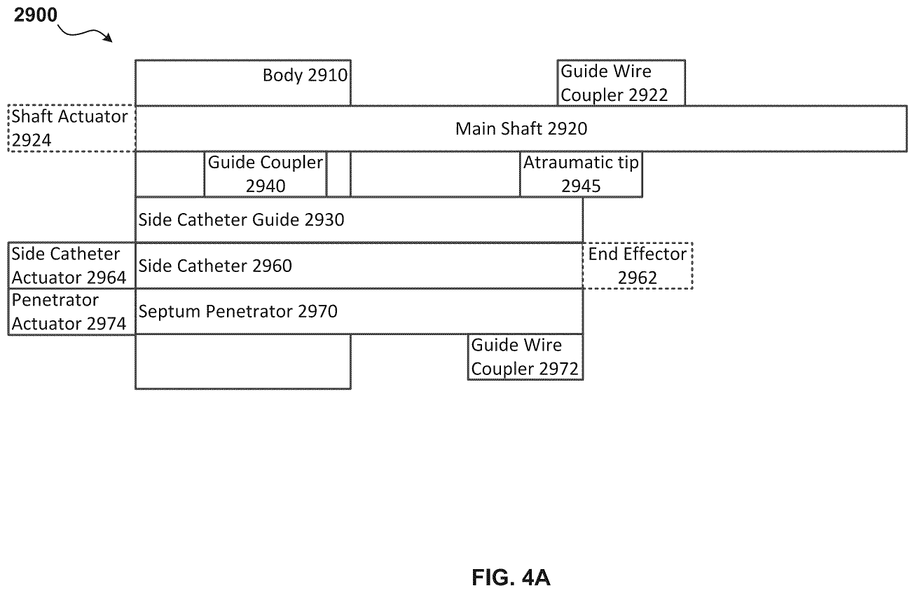

FIG. 3 illustrates a method 200 of using the septal puncture device 100 to access a left atrium of a heart of a patient, according to an embodiment. At 201, the guide wire GW1 is inserted through the IVC, across the RA, and into SVC of the heart (e.g., via a femoral vein puncture and through the patient's vasculature disposed between the femoral vein puncture site and the IVC). At 202, the septal puncture device 100 is delivered over the guide wire GW1 until a distal end of a main shaft 110 is disposed within the SVC. At 204, the GSA 150 is actuated to laterally deflect and direct the side catheter guide 130 towards the FO. Optionally, at 206, the main shaft 110 and the side catheter guide 130 are selectively positioned (e.g., translated or rotated) relative to the FO. Optionally, at 208, the end effector 162 is deployed. At 210, the end effector 162 (or distal end of side catheter) is advanced against and into contact with the FO (e.g., to tent the FO). Optionally, at 212, the end effector 162 (or distal end of side catheter 130) is visualized from outside the patient, and if necessary, the main shaft 110 or the side catheter guide 130 are adjusted to selectively reposition the end effector 162 (or distal end of side catheter 130) relative to the FO.

At 214, the septum penetrator 170 is advanced through the FO and into the LA. Optionally, at 216, visualization techniques are used to confirm crossing of the septum penetrator 170 into the LA. At 220, the guide wire GW2 is advanced relative to the septum penetrator 170 and into the LA or the septum penetrator 170 is withdrawn relative to the septum penetrator 170, thereby leaving a portion of the guide wire GW2 in the LA. At 222, the septum penetrator 170 is withdrawn, the end effector 162 is optionally withdrawn, the main shaft 120 is withdrawn, the guide actuator 150 is deactuated, and the device 100 is withdrawn over the guide wire GW1 and removed from the patient.

Although not shown, in some embodiments, any of the main shafts described herein can define a channel through which an intra-cardiac echo can be disposed or slidably coupled to assist in navigation through the patient.

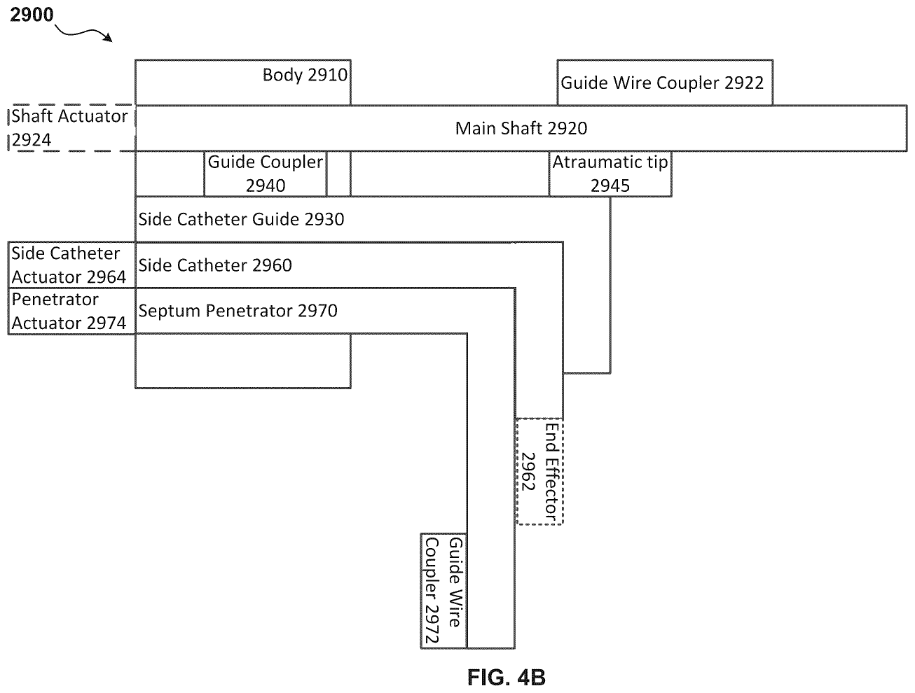

In general, devices such a catheters introduced into the vasculature of a patient carry a risk of inadvertent trauma to the patient's vascular wall and/or associate tissues, organs, etc. A sharp edge of a device, for example, could lacerate a vascular wall. In the context of this disclosure, a main shaft (e.g., main shaft 120), and/or a side catheter guide (e.g., side catheter guide 130) of a septum puncture device could exert a traumatic force against a wall of a curved or tortuous vessel. This could be of particular concern, for example, when a relatively stiff main shaft is used (e.g., for purposes of providing stability between the IVC and SVC). Further, having a side catheter guide adjacent the main shaft may present additional similar risks. To address such risks, any suitable portions of the septum puncture devices described herein can have atraumatic designs. FIGS. 4A and 4B illustrate such a septum puncture device 2900, according to an embodiment. Similar to or the same as described with respect to the septum puncture devices described herein, the septum puncture device 2900 can be used to access a left side of the heart (e.g., left atrium) from the right side of the heart (e.g., right atrium) and to deliver a guidewire to the left side of the heart. The septum puncture device 2900 can be constructed the same as or similar to, and can function the same as or similar to, any of the septum puncture devices described herein (e.g., septum puncture device 100). Thus, portions of the septum puncture device 2900 are not described in further detail herein.

As shown in FIG. 4A, the septum puncture device 2900 includes a body 2910 coupled to a main shaft 2920, a side catheter guide 2930, a side catheter 2960 (with an optional end effector 2962 extending therefrom), a septum penetrator 2970, and an atraumatic tip 2945. The main shaft 2920 is coupled to the side catheter guide 2930 via a guide coupler 2940, the side catheter guide 2930 is coupled to the side catheter 2960, and the side catheter 2960 is coupled to the septum penetrator 2970, as shown in FIG. 4A. The side catheter guide 2930 is configured to define a pathway through or across which the side catheter 2960 can travel (e.g., be advanced and/or withdrawn). Said another way, and as described in further detail herein, the side catheter guide 2930 can be manipulated (e.g., actuated from a delivery state to a deployed state) to guide the side catheter 2960 in a desired direction (the actuated or deployed state of the side catheter guide 2930 is shown in FIG. 4B), e.g., towards the left atrium.

The atraumatic tip 2945 is configured to protect the patient from inadvertent trauma caused by a portion of the side catheter guide 2930, such as, for example, a distal end portion of the side catheter guide 2930, which during insertion is guided into the patient's vasculature by the main shaft 2920. The atraumatic tip 2945 can be formed of any suitable material and can have any suitable shape. In some implementations, the atraumatic tip 2945 can be mounted on and/or coupled to the main shaft 2920. In some implementations, for example, the atraumatic tip 2945 be a nosecone (e.g., a blunt nosecone) mounted on and/or coupled to the main shaft 2920, with a tapered leading edge and a radiused trailing edge (e.g., such that the atraumatic tip 2945 is void of sharp edges). In some implementations, the atraumatic tip 2945 can be asymmetrically mounted on or coupled to the main shaft 2920 such that the atraumatic tip 2945 protects a distal end portion of the side catheter guide 2930 while limiting an overall diameter, cross-sectional area, and/or profile of the main shaft 2920 and side catheter guide 2930.

In some implementations, the atraumatic tip 2945, the main shaft 2920, and/or the body 2910 can be monolithically formed, while in other implementations, the atraumatic tip 2945, the main shaft 2920, and/or the body 2910 can be formed separately and then coupled to one another. In some such implementations, for example, the body 2910 and the atraumatic tip 2945 can be monolithically formed. Further to this example, the body 2910 and the atraumatic tip 2945 can define a lumen through which the main shaft 2920 can be slidably disposed. Further, the body 2910 and atraumatic tip 2945 can be configured to extend distally relative to the main shaft 2920 as far as desired; for example, the body 2910 and the atraumatic tip 2945 can have a distal end terminating proximal to the distal end of the main shaft 2920, at the distal end of the main shaft 2920, or distal to the distal end of the main shaft 2920. Further, the monolithically formed body 2910 and atraumatic tip 2945 can define a lateral opening to allow for the side catheter guide 2930 to extend and/or laterally deflect (e.g., away from the septum) and a lateral opening to through which the distal end of the side catheter guide 2930, the side catheter 2960, the septum penetrator 2970, and/or the guide wire (e.g., to be delivered to the left atrium), can extend.

In some implementations, the main shaft 2920 and the atraumatic tip 2945 can be monolithically formed, and define a lumen through which the side catheter guide 2930 (and a guide wire, for example) can be disposed. In some such implementations, the main shaft 2920/atraumatic tip 2945 can include a guide coupler coupler (not shown) configured to facilitate coupling of the main shaft 2920/atraumatic tip 2945 to the guide coupler 2940. The guide coupler coupler can be any suitable mechanism or feature suitable to secure the guide coupler 2940 to the main shaft 2920/atraumatic tip 2945. As an example, the guide coupler can be a plurality of lateral apertures, slots, or the like defined by the main shaft 2920/atraumatic tip 2945 and configured to receive a portion of the guide coupler 2940.

In some implementations, the atraumatic tip 2945 can have a distal end configured to be spaced distal to the guide coupler 2940, a proximal end extending towards the body 2910, and two lateral openings disposed between the distal end and the proximal end; one lateral opening configured to allow for the side catheter guide 2930 to extend and/or laterally deflect (e.g., away from the septum) and the other lateral opening configured to provide access through which the distal end of the side catheter guide 2930, the side catheter 2960, the septum penetrator 2970, and/or the guide wire (e.g., to be delivered to the left atrium), can extend.

As described in further detail herein in other embodiments, the guide coupler 2940 can couple the side catheter guide 2930 to the main shaft 2920 to minimize or prevent relative translational movement between the main shaft 2920 and the side catheter guide 2930, but to allow relative rotational movement between the main shaft 2920 and the side catheter guide 2930, as illustrated schematically in FIG. 4B. In this manner, the guide coupler 2940 can facilitate transition of the side catheter guide 2930 from a delivery configuration (e.g., parallel to or substantially parallel to the main shaft 2920), e.g., for insertion through the patient's vasculature and into the RA, to a deployed configuration such that a distal end of the side catheter guide 2930 is deflected angularly and/or laterally relative to the main shaft 2920, e.g., towards the patient's left atrium (e.g., the FO of the atrial septum).

The atraumatic tip 2945 can be configured to facilitate such transition of the side catheter guide 2930 into its deployed configuration. In some implementations, for example, the atraumatic tip 2945 can define one or more apertures, lateral openings, and/or slots through which the distal end portion of the side catheter guide 2930 can angularly and/or laterally deflect, and/or through which a portion of the side catheter guide 2930 that is proximal to the distal end portion of the side catheter guide 2930 can extend and/or deflect (e.g., the proximal portion being one a first side of a central axis of the shaft while the distal portion is on a second side of the central axis opposite the first side of the central axis. In this manner, the side catheter guide 2930 is shielded prior to deployment, and free to deflect and assume an increased profile during deployment.

In some implementations, the entire atraumatic tip 2945 can be disposed distal to the guide coupler 2940, while in some implementations, the atraumatic tip 2945 can extend across and proximally beyond the guide coupler 2940.

The atraumatic tip 2945 can be of any suitable size. For example, in some implementations, the atraumatic tip 2945 can have an outer diameter of about 14 F. As another example, in some implementations, the atraumatic tip 2945 can have a length in a range of about 1 mm to about 150 mm. In some implementations, the atraumatic tip 2945 can have a length of about 10-30 times its diameter; such a length could be, for example, 75 mm, 100 mm, 150 mm, or any value therebetween.

In some implementations, the atraumatic tip 2945 can include a radiopaque material and/or marker (e.g., a band and/or a groove) such that the atraumatic tip 2945 can be visualized when within the heart from outside the patient under any suitable imaging modality (e.g., fluoroscopy, echocardiography, etc.), to facilitate an operator in deploying the side catheter guide 2930 and/or the side catheter 2960.

Further as shown in FIG. 4A, the septum puncture device 2900 includes a guide wire coupler 2922 configured to couple the main shaft 2920 to a guide wire (not shown in FIG. 4A) to facilitate delivery of the septum puncture device 2900 into a patient (e.g., through the vasculature of the patient) and to the patient's heart, and a guide wire coupler 2972 configured to couple a guide wire (not shown in FIG. 4A) to the septum penetrator 2970, to facilitate delivery of that guide wire to the left side of the heart (e.g., the left atrium).

Further as shown in FIG. 4A, the septum puncture device 2900 optionally includes a shaft actuator 2924 operably coupled to the main shaft 2920 and configured to actuate the main shaft 2920 to advance or withdraw the main shaft 2920 relative to the body 2910. The septum puncture device 2900 further includes (1) a side catheter actuator 2964 operably coupled to and configured to actuate the side catheter 2960 to advance or withdraw the side catheter 2960, thereby transitioning the side catheter 2960 between a delivery configuration and a deployed configuration (the side catheter 2960 shown in an actuated or deployed configuration in FIG. 4B), and (2) a septum penetrator actuator (or “penetrator actuator”) 2974 to actuate the septum penetrator 2970 to advance or withdraw the septum penetrator 2970, thereby transitioning the septum penetrator between a delivery configuration and a deployed configuration (the septum penetrator 2970 shown in an actuated or deployed configuration in FIG. 4B), as described in further detail herein.

Further as shown in FIG. 4A, the septum puncture device 2900 optionally includes an end effector 2962 coupled to and extending distally from the side catheter 2960. The end effector 2962 is configured to facilitate subsequent puncture through a target puncture location, such as, for example, the FO of the septum of the heart. The end effector 2962 can be configured, for example, to contact or tent the FO, as described in further detail herein. Such contact or tenting of the FO can, for example, reduce or minimize the force required to penetrate the FO and/or provide for improved force distribution to the FO. The end effector 2962 can be configured to prevent inadvertent puncturing of and/or damage to the FO with the end effector 2962.

Each of the main shaft 2920, the guide wire coupler 2922, the side catheter guide 2930, the guide coupler 140, the side catheter 2960, the septum penetrator 2970, and the guide wire coupler 2972 are translatable (e.g., distally advanceable and/or extendable, and proximally withdrawable and/or retractable) relative to the body 2910. The side catheter 2960 is translatable relative to the side catheter guide 2930, and the septum penetrator 2970 is translatable relative to the side catheter 2960, as described in further detail herein.

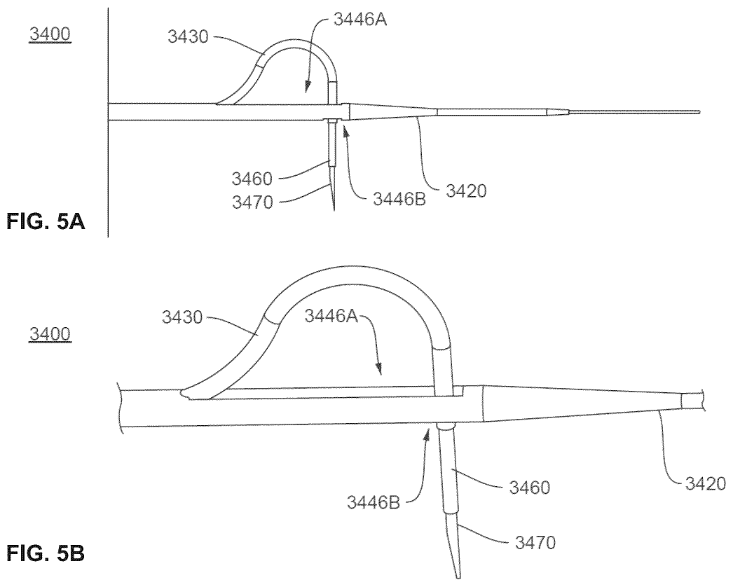

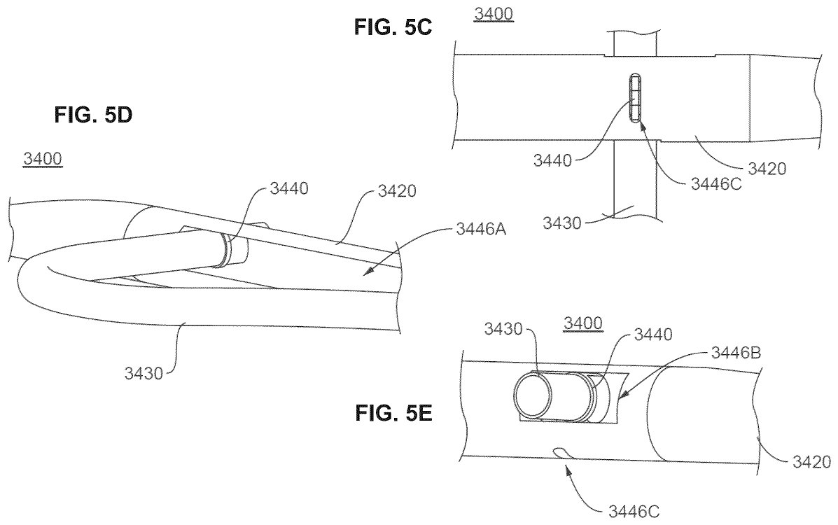

Although various atraumatic tips described herein are shown as a component and/or material that is formed separately and then coupled to the main shaft, in some embodiments, the functionality of an atraumatic tip (e.g., the atraumatic tip 3245) can be incorporated into and provided by the main shaft. FIGS. 5A-5E illustrate a portion of a septum puncture device, in various views, in a deployed configuration, according to such an embodiment.

Similar to other septum puncture devices described herein, the septum puncture device 3400 can be used to access a left side of the heart (e.g., left atrium) from the right side of the heart (e.g., right atrium) and to deliver a guidewire to the left side of the heart. The septum puncture device 3400 can be constructed the same as or similar to, and can function the same as or similar to, any of the septum puncture devices described herein. Thus, portions of the septum puncture device 3400 are not described in further detail herein.

In this embodiment, the septum puncture device 3400 includes a main shaft 3420 and a side catheter guide 3430 coupled to the main shaft 3420 via a guide coupler 3440 (shown in FIGS. 5C-5E). A portion of the side catheter guide 3430 disposed proximal to the guide coupler 3420 is slidably disposed within a lumen defined by the main shaft 3420, and a portion of the side catheter guide 3430 disposed distal to the guide coupler is disposed within and deflectable relative to the lumen of the main shaft 3420. Slidably disposed within the side catheter guide 3430 is a side catheter 3460, and slidably disposed within the side catheter 3460 is a septum penetrator 3470 (as shown in FIGS. 5A and 5B). Although not shown in this embodiment, as can be the case in any of the embodiments described herein, in some implementations, the septum puncture device (e.g., including the septum puncture device 3400) can include an end effector (e.g., similar to or the same as in form and/or function as any of the end effectors described herein). As shown best in FIG. 5B, the main shaft 3420 defines a first slot 3446A and a second slot 3446B (both of which are in communication with the lumen of the main shaft 3420). During deployment, the side catheter guide 3430 can deflect and extend through and beyond the first slot 3446A and the second slot 3446B, similar to as described above with respect to the septum puncture device 3200 and the septum puncture device 3330.

The main shaft 3420 further includes a guide coupler 3446C that is configured to promote coupling between the guide coupler 3440 and the main shaft 3420. In this embodiment, the guide coupler 3446C is formed of two apertures defined within the main shaft 3420 and configured to receive a portion of the guide coupler 3446 (see e.g., FIG. 5C). In this manner, the side catheter guide 3430 can be secured to the main shaft 3420 via the guide coupler 3446, such that relative rotational movement between the main shaft 3420 and the side catheter guide 3420 is promoted, but relative translational movement between the same is limited or prevented.

In some embodiments, a needle can be aimed at a specific region of the FO for puncture. The FO can be divided into quadrants, for example, in which a puncture in each quadrant is advantageous for a specific procedure. The needle can thereby be aimed to puncture slightly superior, posterior, and 3.5 cm-4.5 cm above the mitral valve for a MitraClip device, or to puncture posterior and slightly inferior within the FO for typical left atrial appendage occlusion devices. After successful puncture and insertion of a guidewire, the septum puncture device can be completely removed to make way for any suitable instrument or device to be guided into the left atrium of the heart to perform a desired procedure, such as atrial fibrillation ablation, left atrial appendage closure, and valve replacements.

Various embodiments described herein include a side catheter guide configured to transition from a delivery configuration to a deployed configuration in response to a distal force applied to a portion of the side catheter guide that is disposed proximal to the guide coupler (e.g., a distal force applied at the handle). In some implementations of such embodiments described herein, instead of or in addition to such distal force, a proximal force can be applied to the main shaft (e.g., proximal the guide coupler) to cause similar deployment of the side catheter guide. Said another way, deployment of the side catheter guide can be accomplished merely by relative movement between the main shaft and the side catheter guide, which can include a proximal force applied to the main shaft and/or a distal force applied to the side catheter guide.

In various embodiments described herein, a side catheter guide is deflected such that a distal end portion of the side catheter guide angularly and/or laterally deflects about 90 degrees relative to a central axis of a main shaft to which the side catheter guide is coupled. In any of the embodiments described herein, in some implementations, such deflection can be greater than or less than 90 degrees. In such implementations, the deflection may be less than less than about 90 degrees, such as, for example, about 15 degrees, about 30 degrees, about 45 degrees, about 60 degrees, about 75 degrees, or any degrees therebetween. In some implementations, the deflection may be about 75 degrees to about 85 degrees, e.g., about 80 degrees. In even further implementations, the deflection may be greater than about 90 degrees, such as, for example, about 95 degrees, about 105 degrees, about 110 degrees, about 115 degrees, about 120 degrees, about 135 degrees, or any degrees therebetween. In yet further implementations, the deflection may be from about 50 degrees to about 90 degrees.

In various embodiments described herein, a side catheter guide is deflected such that a distal end portion of the side catheter guide angularly and/or laterally deflects relative to a central axis of the a main shaft to which the side catheter guide is coupled (and/or relative to a target tissue, such as an atrial septum). In any of the embodiments described herein, in some implementations, deflection of the side catheter guide can be operator-selectable, meaning that an operator of the septum puncture device can select a particular amount or angle of deflection from among multiple available amounts or angles of deflection. In such implementations, for example, a side catheter guide can be configured to deflect a first amount or angle and a different, second amount or angle, such that an operator can selectively deflect the side catheter guide as desired (e.g., based on a particular patient's anatomy, and/or the particular procedure(s) being performed). In this manner, a septum puncture device can have multiple deployed configurations, each having varying amounts/angles of deflection.

Further, in some embodiments, the deflection selected by the operator can be subsequently fixed and/or temporarily locked in place, such that the selected deflection remains during subsequent steps, such as, for example, distal extension of a side catheter and/or puncture member, and subsequent puncture of the target tissue. Such fixation can be employed in any suitable manner. As an example, a proximal end portion of the side catheter guide can be slidably fixed (e.g., to a body and/or a handle assembly).

Various embodiments described herein include a GSA or balloon configured to transition between a delivery configuration and a deployed configuration. In some implementations of any of the embodiments described herein, one or more GSAs or balloons can be covered partially or completely with a mesh made from any suitable material (e.g., nylon, polymer, etc.). The mesh, coupled to a balloon, for example, can facilitate a preferred, predefined shape of the balloon when inflated, or can facilitate the step or steps of inflating the balloon by, e.g., providing additional stability.

Although various embodiments described herein focus on using a puncture device to puncture a septum of a heart, the functionality provided by various puncture devices described herein can be desirable in other procedures and in other parts of a patient. For example, many procedures exist in which it would be desirable to be able to provide a stable, precise, safe, and repeatable lateral puncture. In some instances, for example, any of the puncture devices described herein could be used to facilitate a tricuspid annuloplasty. The puncture device, for example, could be arrange such that a central axis of its main shaft is parallel to a plane of the tricuspid valve, and so the puncture device could provide lateral or perpendicular access to the annulus of the tricuspid, e.g., to deliver sutures, screws, or other anchoring devices for purposes of a tricuspid annuloplasty.

As another example, the puncture devices described herein could provide a access and a direct vector to a coronary sinus of a heart, to, e.g., insert or deliver a wire, a catheter, a mitral valve repair device, pacemaker leads, etc. into the coronary sinus.

As another example, the puncture devices described herein could be used for delivering therapeutic repair or replacement devices to a mitral valve within a heart. If, for example, a side catheter guide or a side catheter disclosed herein were extended further, and beyond about 90 degrees, the side catheter could be directed into the LA and towards the mitral valve. In some instances, the natural trajectory of the side catheter in some of the embodiments described herein would be angled or directed towards the mitral valve if extended or advanced a suitable distance. For example, as the side catheter assumes its laterally deflected shape or orientation, it may be curved or possess an arc, such that further advancement relative to the main shaft results in the side catheter advancing along such a curvature or arc such that the distal end of the side catheter turns or is further laterally deflected towards the mitral valve. Said another way, in some instances, advancement of the side catheter from its delivery configuration to an advanced/deployed configuration can include the distal end of the side catheter being laterally deflected up to about 180 degrees.

As another example, the puncture devices described herein could incorporate an intracardiac echo catheter to enable accelerate transseptal puncture.

As another example, the puncture devices described herein could be used in connection with cardiac arrest. In such instances, for example, one or more puncture devices could be used in combination with a broad, curved catheter, to enable a guide wire to be directed or delivered from the femoral vein, across the FO, through the mitral valve and out the left ventricular outflow tract (“LVOT”)/aortic valve. In some embodiments a balloon/flow-directed catheter would be advanced across the FO, into the LA, across the mitral valve and then across the LVOT/aortic valve; the balloon, for example, would serve to “flow direct” the catheter out the LVOT and across the aortic valve into the aorta. Once in position, the wire could be used as a track for a small catheter that could provide extracorporeal membrane oxygenation (“ECMO”) and oxygen to the brain. A distal end of the catheter in the aorta would be the outflow, and more proximal ports (e.g., in the RA or the IVC) would be the inflow to the pump.

As another example, the puncture devices described herein could be used in an aorta to facilitate delivery of branch vessel stents, to deliver coils to branch vessels, or to deliver a screen for cerebral embolic protection to the head vessel.

Although various atraumatic tips described herein are shown as a component and/or material that is formed separately and then coupled to the main shaft, in some embodiments, the functionality of an atraumatic tip (e.g., the atraumatic tip 3245) can be incorporated into and provided by the main shaft. FIGS. 5A-5E illustrate a portion of a septum puncture device, in various views, in a deployed configuration, according to such an embodiment.

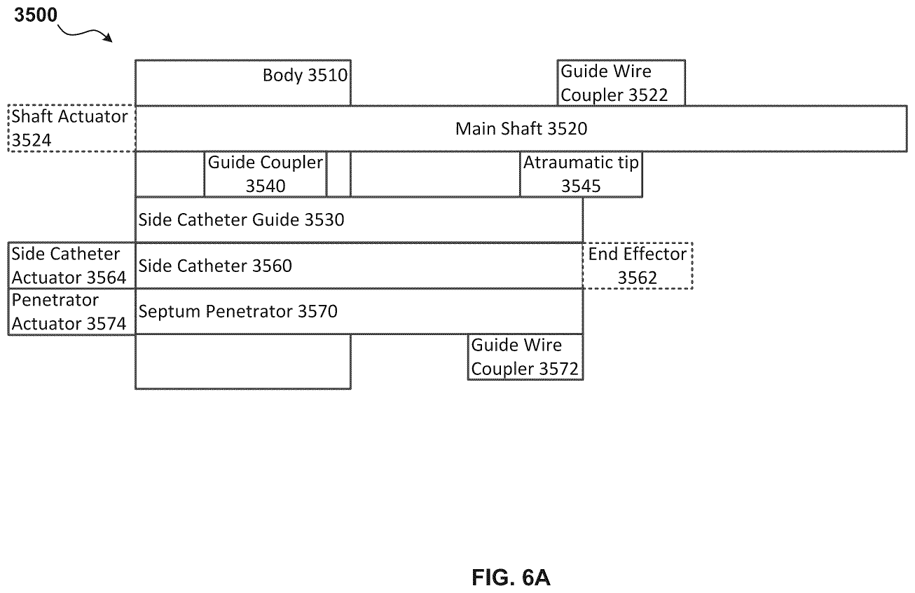

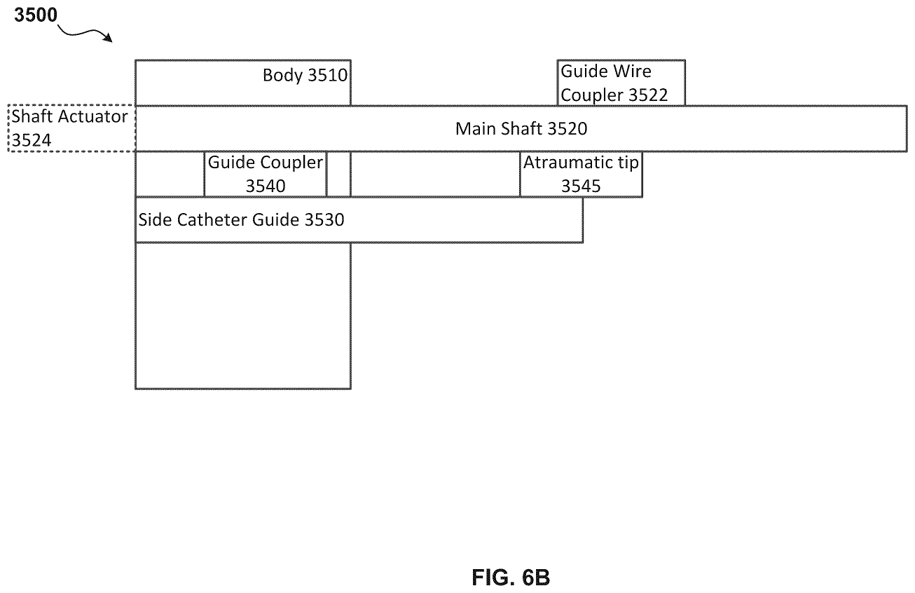

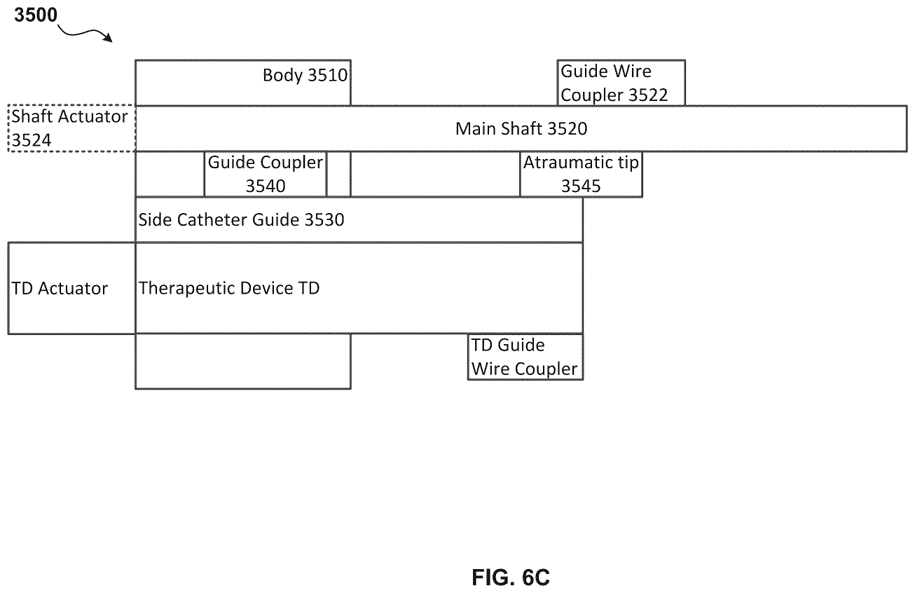

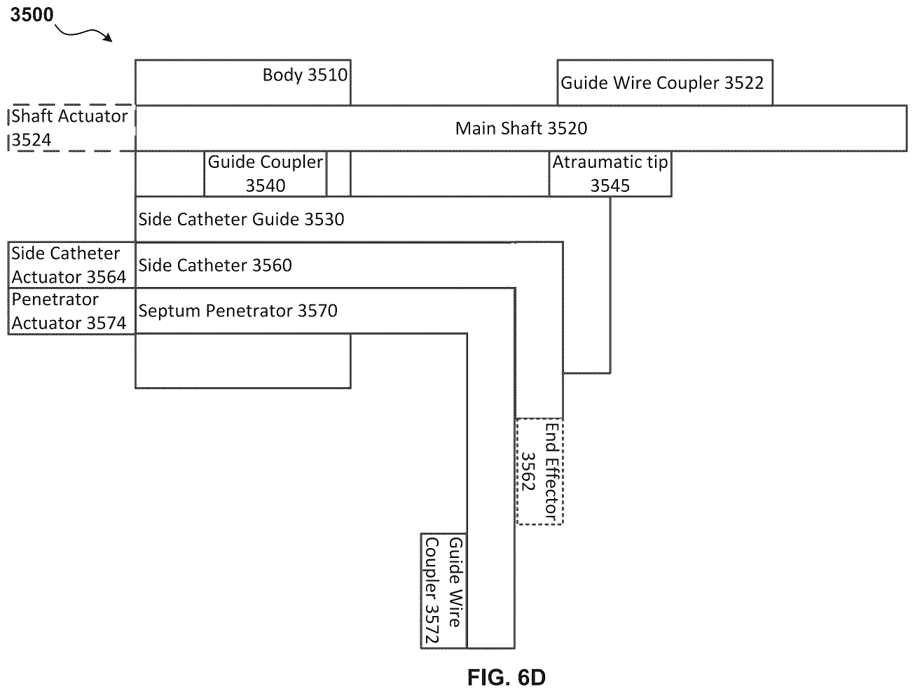

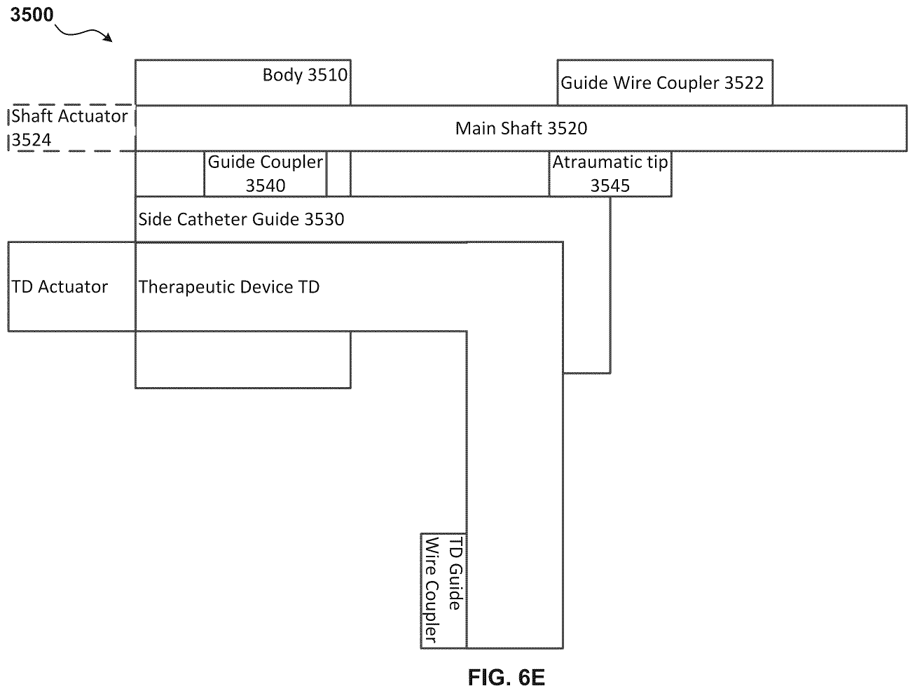

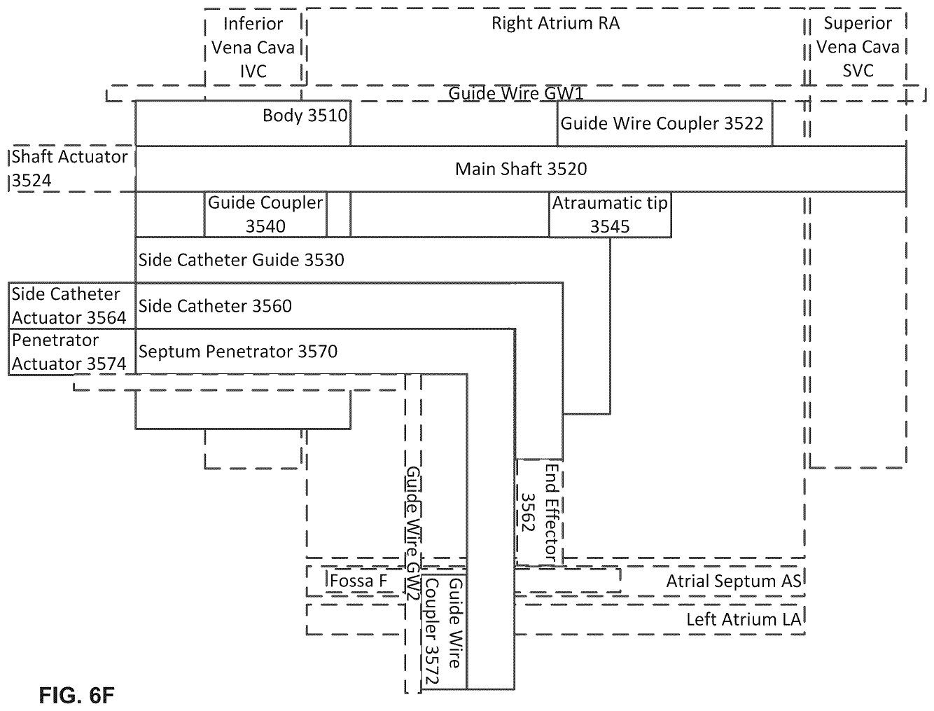

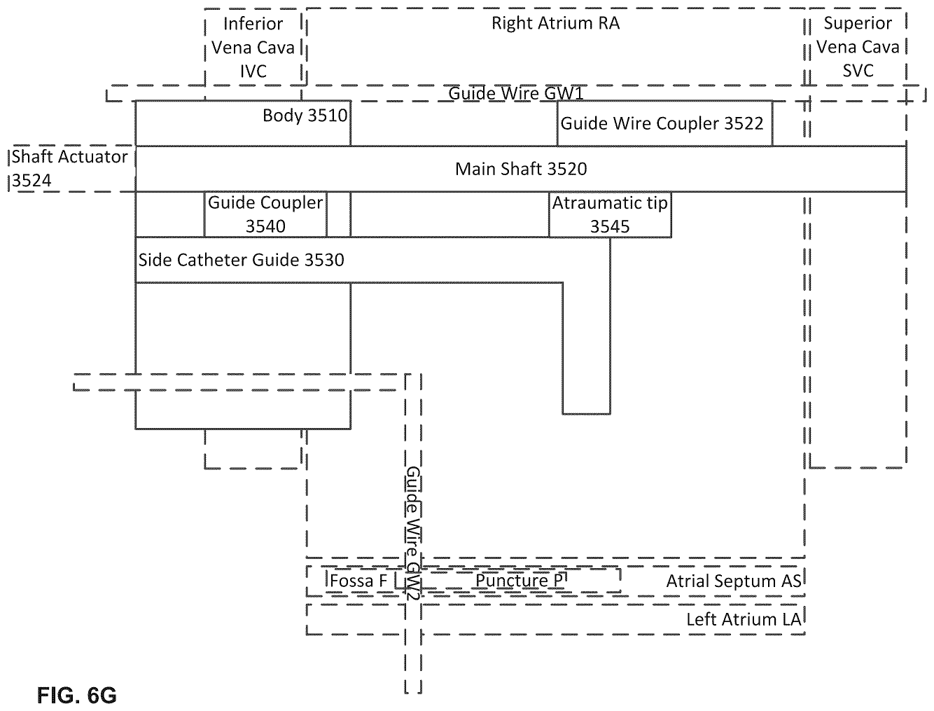

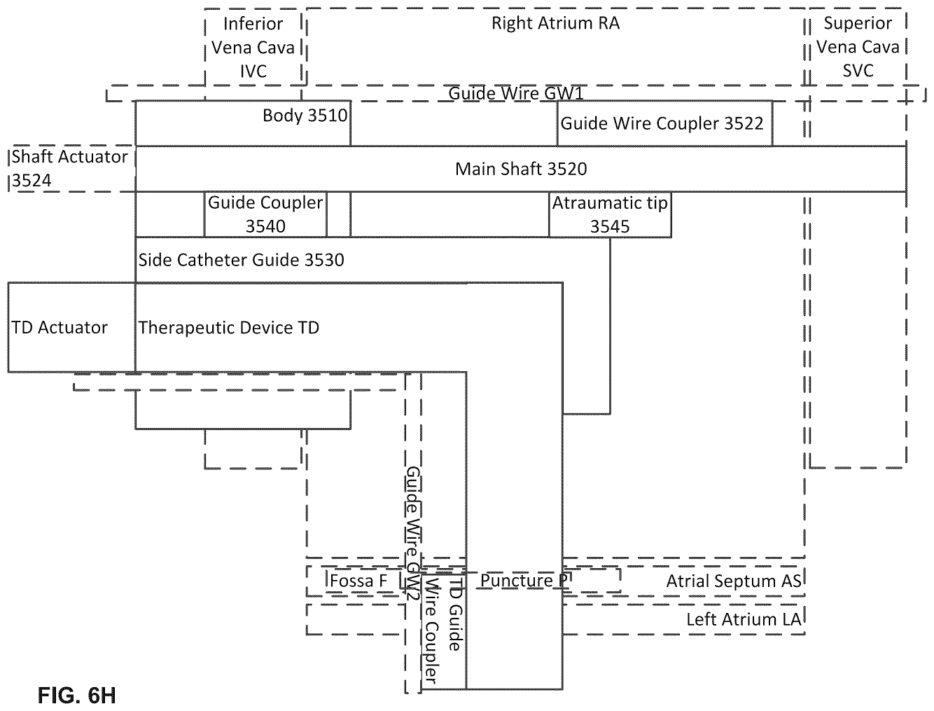

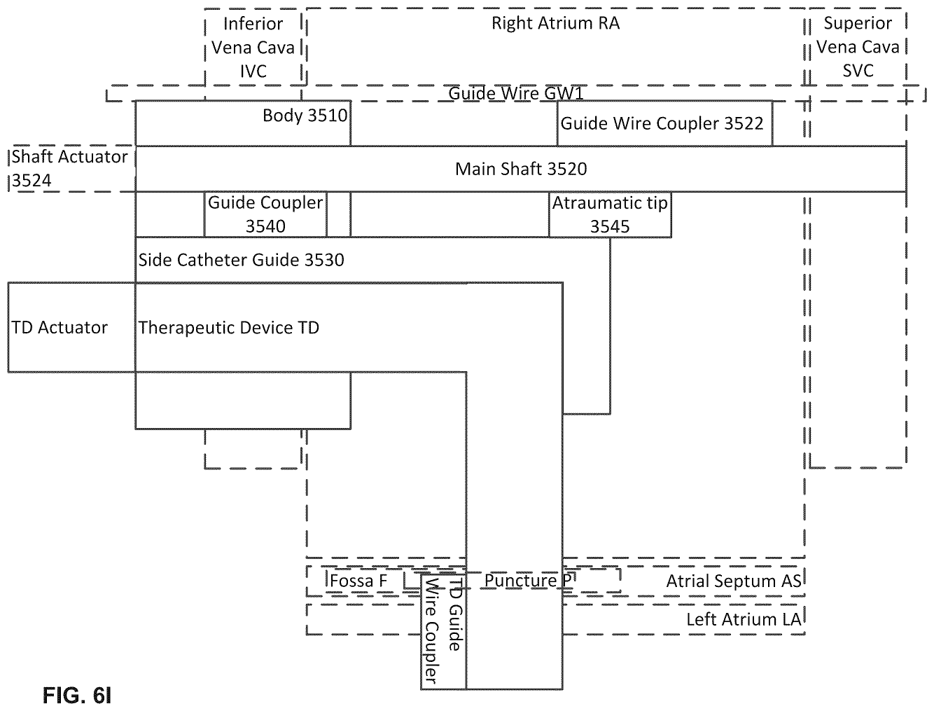

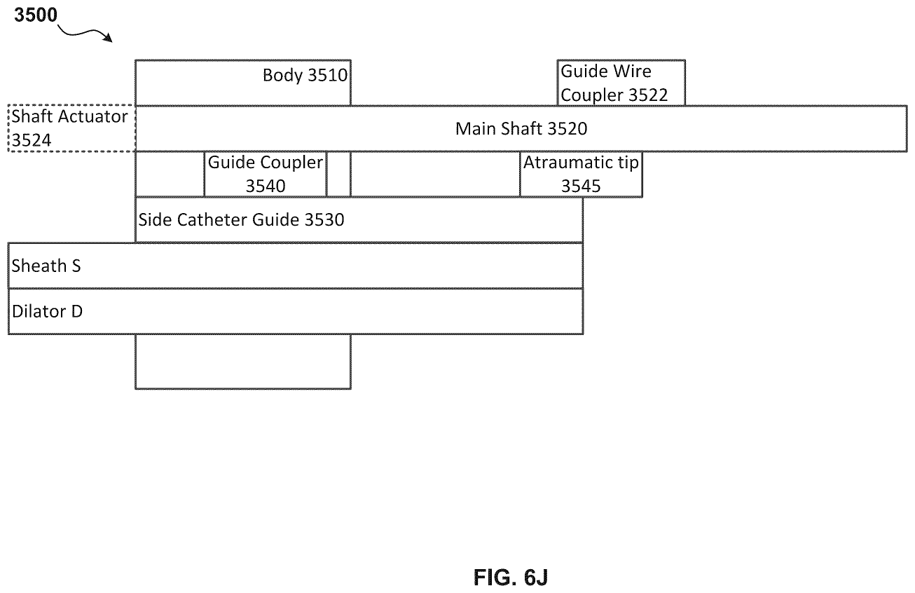

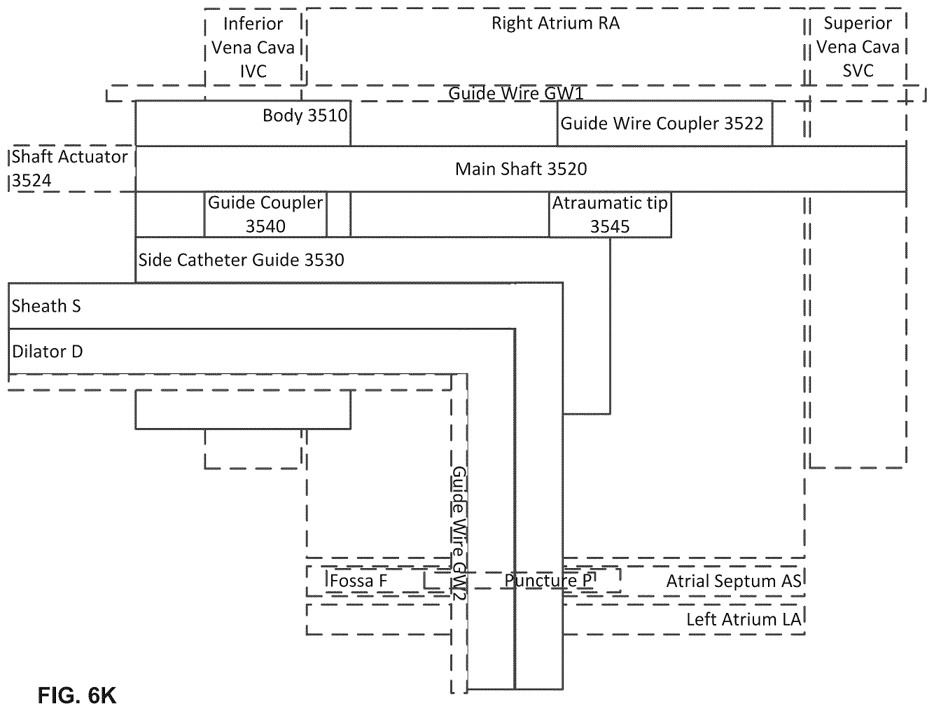

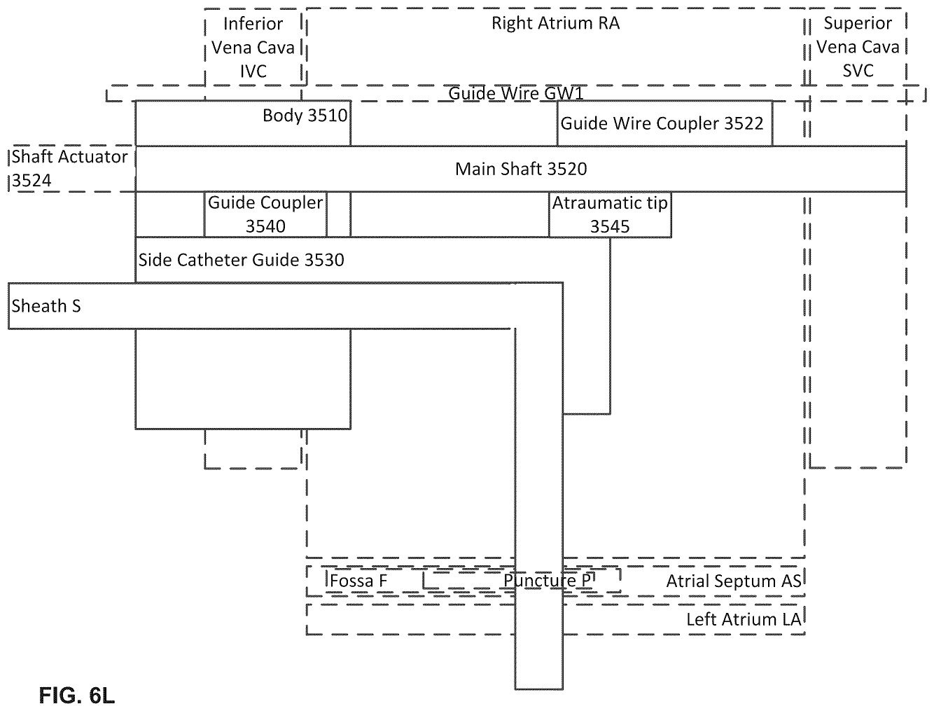

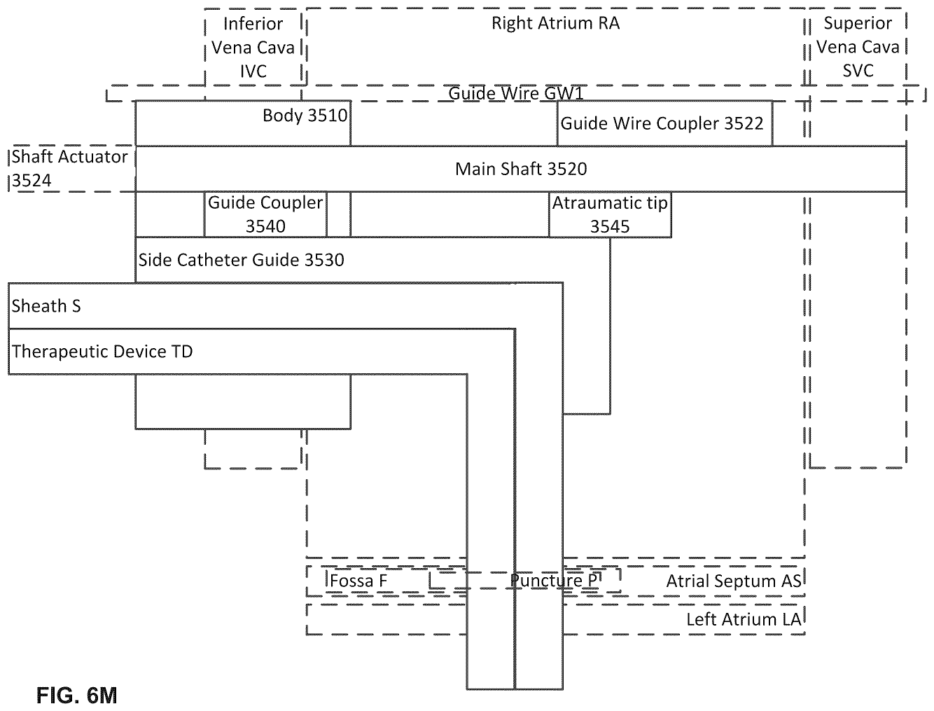

In other embodiments, a septum puncture device can be configured to be usable to assist in the delivery of a therapeutic device across the septum and into the LA (or in any other delivery scenario described above). Such a device may be further configured to be usable to assist in maneuvering the therapeutic device in the LA (or other anatomy) for the balance of the clinical procedure for which the therapeutic device is used. One embodiment of such a septum puncture device is illustrated schematically in FIGS. 6A to 6M. Septum puncture device 3500 is essentially the same as septum puncture device 2900, except for the differences described below, and therefore the following description is abbreviated. However, it should be understood that omitted details can be inferred from the description of septum puncture device 2900 with reference to FIGS. 4A and 4B.

Similar to or the same as described with respect to the septum puncture devices described herein, the septum puncture device 3500 can be used to access a left side of the heart (e.g., left atrium) from the right side of the heart (e.g., right atrium) and to deliver a guidewire to the left side of the heart. The septum puncture device 3500 can be constructed the same as or similar to, and can function the same as or similar to, any of the septum puncture devices described herein (e.g., septum puncture devices 100, 2900).

As shown in FIG. 6A, the septum puncture device 3500 includes a body 3510 coupled to a main shaft 3520, a side catheter guide 3530, a side catheter 3560 (with an optional end effector 3562 extending therefrom), a septum penetrator 3570, and an atraumatic tip 3545. The main shaft 3520 is coupled to the side catheter guide 3530 via a guide coupler 3540, the side catheter guide 3530 is coupled to the side catheter 3560, and the side catheter 3560 is coupled to the septum penetrator 3570, as shown in FIG. 6A. The side catheter guide 3530 is configured to define a pathway through or across which the side catheter 3560 can travel (e.g., be advanced and/or withdrawn). Said another way, and as described in further detail herein, the side catheter guide 3530 can be manipulated (e.g., actuated from a delivery state to a deployed state) to guide the side catheter 3560 in a desired direction (the actuated or deployed state of the side catheter guide 3530 is shown in FIG. 6D), e.g., towards the left atrium.

As described in further detail herein in other embodiments, the guide coupler 3540 can couple the side catheter guide 3530 to the main shaft 3520 to minimize or prevent relative translational movement between the main shaft 3520 and the side catheter guide 3530, but to allow relative rotational movement between the main shaft 3520 and the side catheter guide 3530, as illustrated schematically in FIG. 6D. In this manner, the guide coupler 3540 can facilitate transition of the side catheter guide 3530 from a delivery configuration (e.g., parallel to or substantially parallel to the main shaft 3520), e.g., for insertion through the patient's vasculature and into the RA, to a deployed configuration such that a distal end of the side catheter guide 3530 is deflected angularly and/or laterally relative to the main shaft 3520, e.g., towards the patient's left atrium (e.g., the FO of the atrial septum).

Further as shown in FIG. 6A, the septum puncture device 3500 includes a guide wire coupler 3522 configured to couple the main shaft 3520 to a guide wire (not shown in FIG. 6A) to facilitate delivery of the septum puncture device 3500 into a patient (e.g., through the vasculature of the patient) and to the patient's heart, and a guide wire coupler 3572 configured to couple a guide wire (not shown in FIG. 6A) to the septum penetrator 3570, to facilitate delivery of that guide wire to the left side of the heart (e.g., the left atrium).

Further as shown in FIG. 6A, the septum puncture device 3500 optionally includes a shaft actuator 3524 operably coupled to the main shaft 3520 and configured to actuate the main shaft 3520 to advance or withdraw the main shaft 3520 relative to the body 3510. The septum puncture device 3500 further includes (1) a side catheter actuator 3564 operably coupled to and configured to actuate the side catheter 3560 to advance or withdraw the side catheter 3560, thereby transitioning the side catheter 3560 between a delivery configuration and a deployed configuration (the side catheter 3560 shown in an actuated or deployed configuration in FIG. 6D), and (2) a septum penetrator actuator (or “penetrator actuator”) 3574 to actuate the septum penetrator 3570 to advance or withdraw the septum penetrator 3570, thereby transitioning the septum penetrator between a delivery configuration and a deployed configuration (the septum penetrator 3570 shown in an actuated or deployed configuration in FIG. 6D), as described in further detail herein.

Further as shown in FIG. 6A, the septum puncture device 3500 optionally includes an end effector 3562 coupled to and extending distally from the side catheter 3560. The end effector 3562 is configured to facilitate subsequent puncture through a target puncture location, such as, for example, the FO of the septum of the heart. The end effector 3562 can be configured, for example, to contact or tent the FO, as described in further detail herein. Such contact or tenting of the FO can, for example, reduce or minimize the force required to penetrate the FO and/or provide for improved force distribution to the FO. The end effector 3562 can be configured to prevent inadvertent puncturing of and/or damage to the FO with the end effector 3562.

Each of the main shaft 3520, the guide wire coupler 3522, the side catheter guide 3530, the guide coupler 140, the side catheter 3560, the septum penetrator 3570, and the guide wire coupler 3572 are translatable (e.g., distally advanceable and/or extendable, and proximally withdrawable and/or retractable) relative to the body 3510. The side catheter 3560 is translatable relative to the side catheter guide 3530, and the septum penetrator 3570 is translatable relative to the side catheter 3560, as described in further detail herein.

Septum puncture device 3500 is configured so that side catheter 3560 is releasably coupled to side catheter guide 3530, and side catheter 3560, end effector 3562, and septum penetrator 3570 can be collectively withdrawn proximally through side catheter guide 3530 and removed from the remainder of septum puncture device 3500, to the state shown in FIG. 6B. The removal of this assembly can be done while the septum puncture device 3500 is in the deployed configuration shown in FIG. 6D, e.g. after the septum puncture device has been used to deliver a guidewire through the septum and into the LA, as shown in FIG. 6F, to the state shown in FIG. 6G. Septum puncture device 3500 is also configured so that the internal lumen of side catheter 3530 is large enough to accommodate a therapeutic device TD, so that therapeutic device TD can be inserted into, and delivered distally through, the lumen, as shown in FIG. 6C (in the delivery configuration) and 6E (in the deployed configuration) and into the left atrium LA through the puncture P in the fossa F of the atrial septum AS, as shown in FIG. 6H. Alternatively, or additionally, septal puncture device 3500 can be configured so that a sheath S can be introduced into, and delivered distally through, the lumen, along with a dilator D disposed within the sheath S, as shown in FIG. 6J (in the delivery configuration) and 6K (in the deployed configuration, delivered distally into the left atrium LA through the puncture P in the fossa F of the atrial septum AS). The dilator D can be delivered over the guidewire GW2 that has been inserted through the puncture P (FIG. 6K). The dilator S and guidewire GW2 can be withdrawn proximally and removed from side catheter guide 3530, leaving only the sheath S extending through puncture P (dilated by dilator D) (FIG. 6L). Therapeutic device TD can then be delivered into the left atrium LA through sheath S (FIG. 6M).

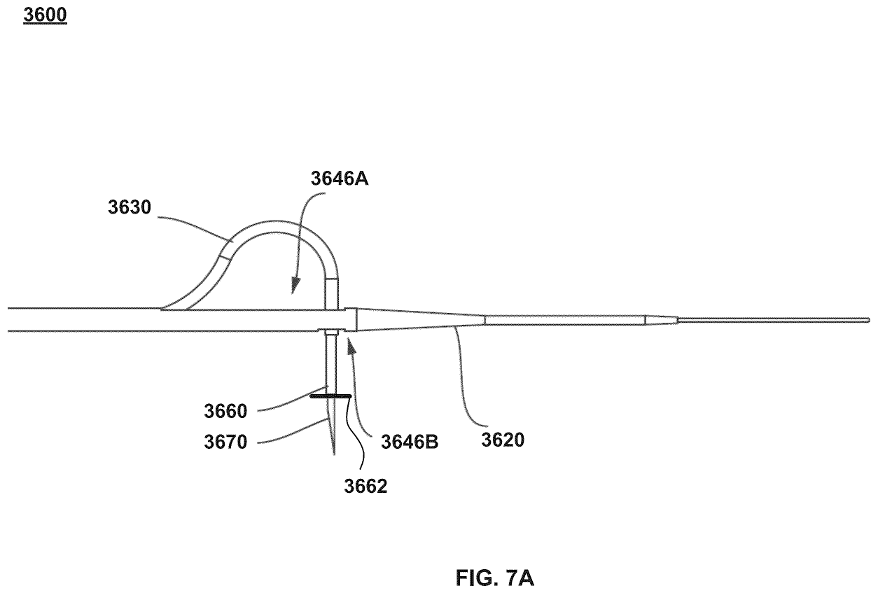

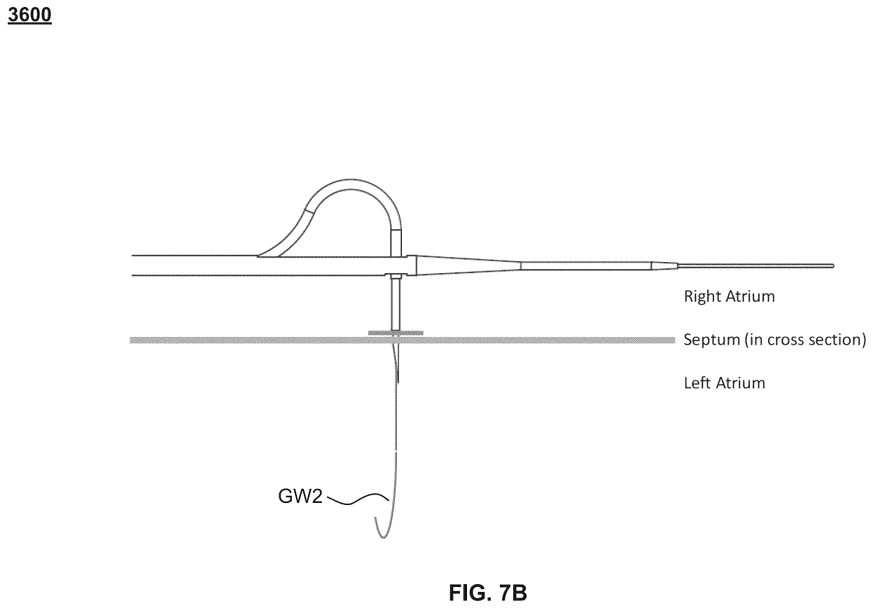

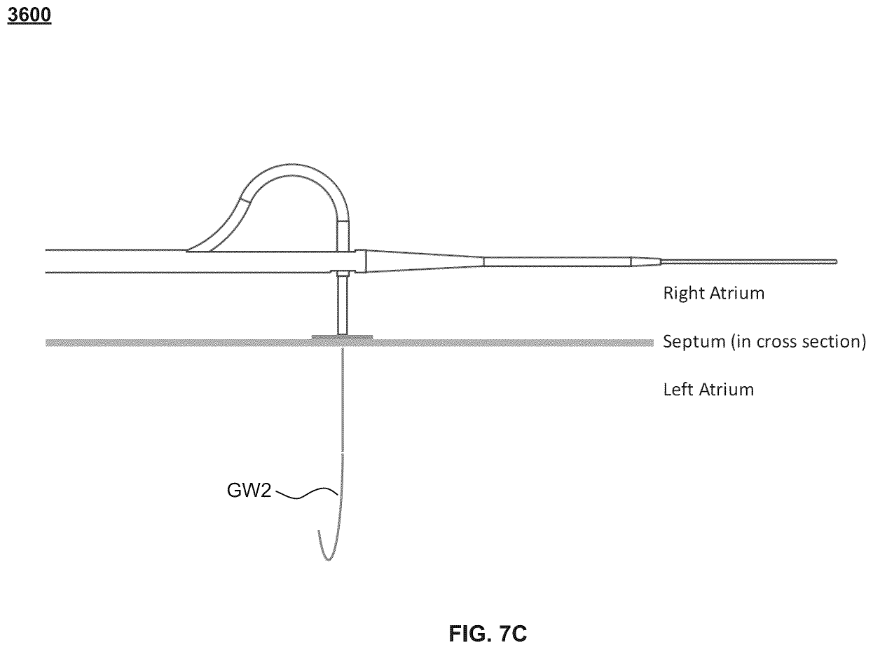

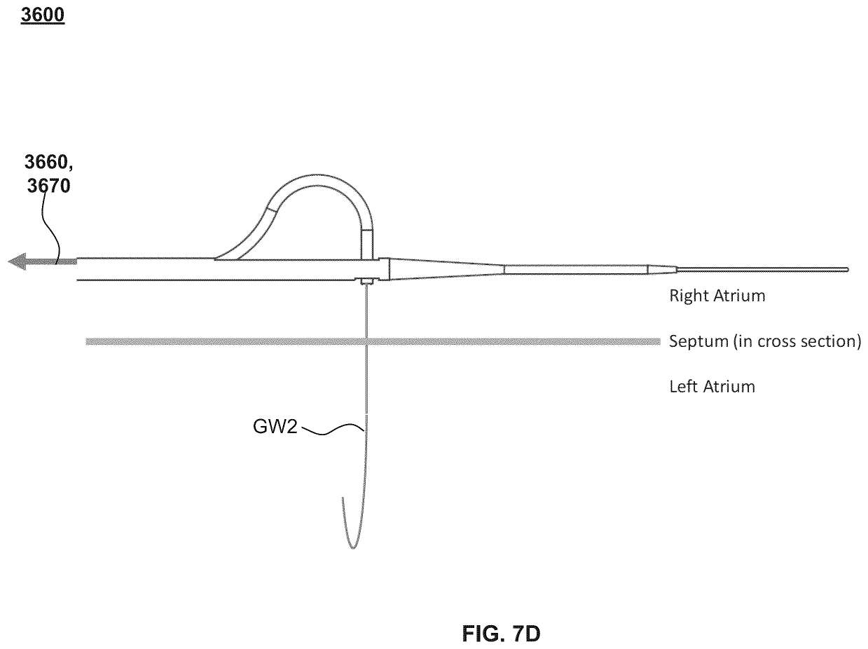

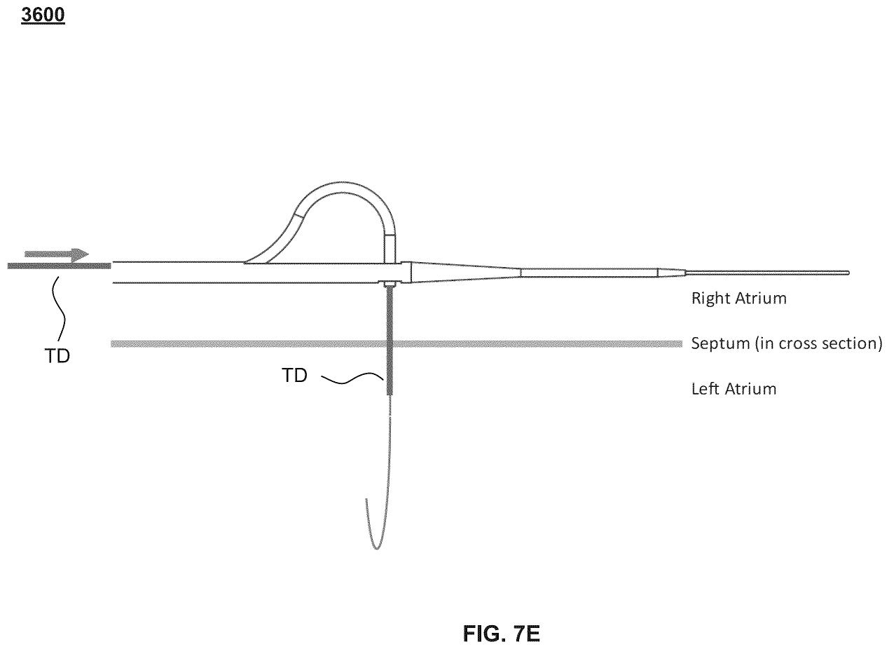

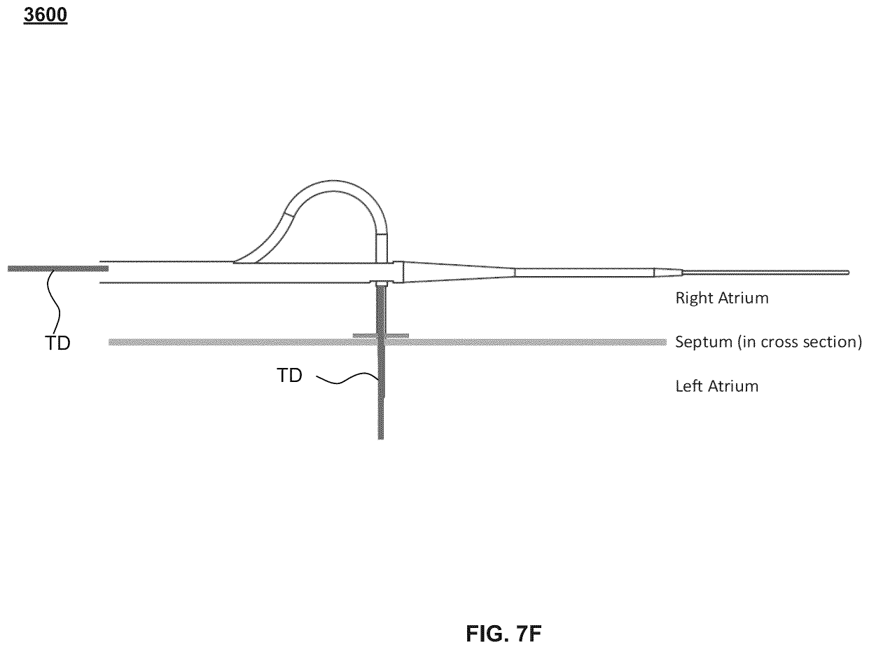

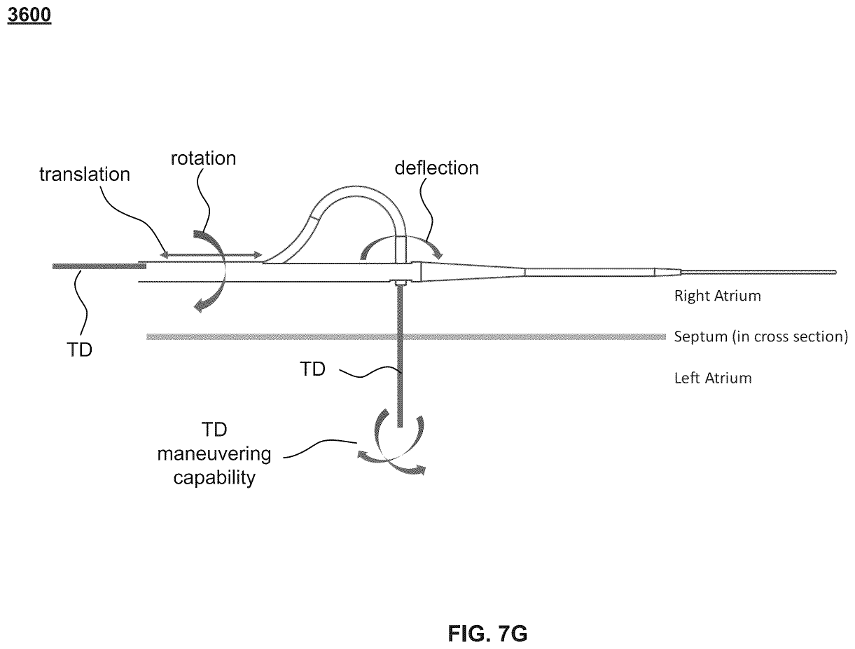

An embodiment of a septum puncture device is illustrated in FIG. 7A. Septum puncture device 3600 is essentially the same as septum puncture device 3400, except for the differences described below, and therefore the following description is abbreviated, and made with reference to a single view of the device. However, it should be understood that omitted details can be inferred from the description of septum puncture device 3400 with reference to FIGS. 5A to 5E. As described in more detail below, septum puncture device 3600 is also one implementation of the septum puncture device 3500 illustrated schematically in FIGS. 6A to 6M.

Septum puncture device 3600 includes a main shaft 3620 and a side catheter guide 3630 coupled to the main shaft 3620 via a guide coupler. A portion of the side catheter guide 3630 disposed proximal to the guide coupler 3620 is slidably disposed within a lumen defined by the main shaft 3620, and a portion of the side catheter guide 3630 disposed distal to the guide coupler is disposed within and deflectable relative to the lumen of the main shaft 3620. Slidably disposed within the side catheter guide 3630 is a side catheter 3660, and slidably disposed within the side catheter 3660 is a septum penetrator 3670.

Unlike septum puncture device 3400, septum puncture device includes an end effector 3662 disposed at a distal end of side catheter 3660. Additionally, septum puncture device is configured so that side catheter 3660 is releasably coupled to side catheter guide 3630, and side catheter 3660, end effector 3662, and septum penetrator 3670 can be collectively withdrawn proximally through side catheter guide 3630 and removed from the remainder of septum puncture device 3600. The removal of this assembly can be done while the septum puncture device 3600 is in the deployed configuration shown in FIG. 6, e.g. after the septum puncture device has been used to deliver a guidewire through the septum and into the LA. Septum puncture device 3600 is also configured so that the internal lumen of side catheter 3630 is large enough to accommodate a therapeutic device desired to be delivered therethrough.

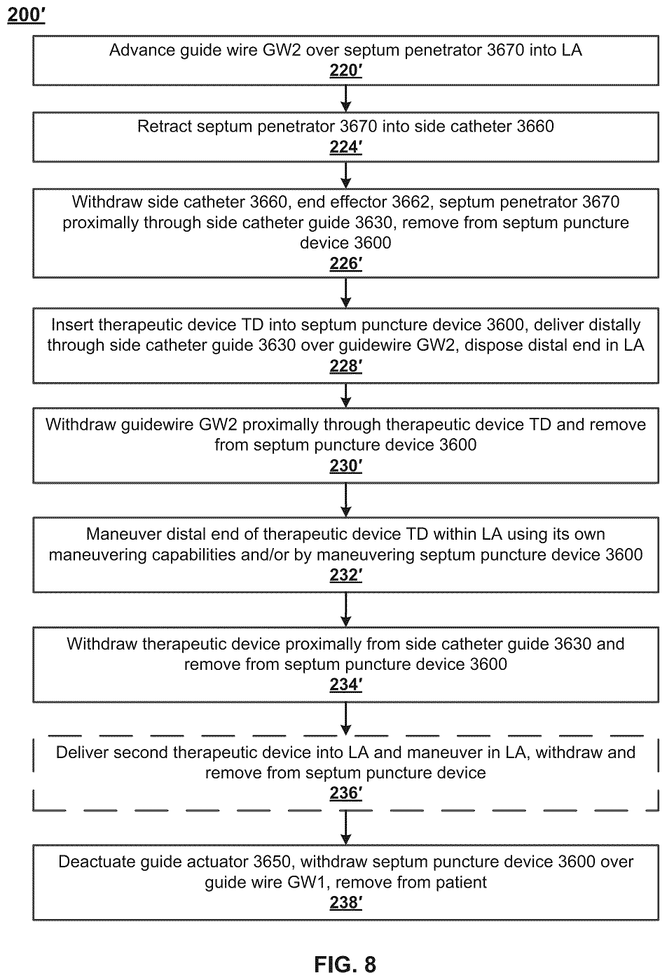

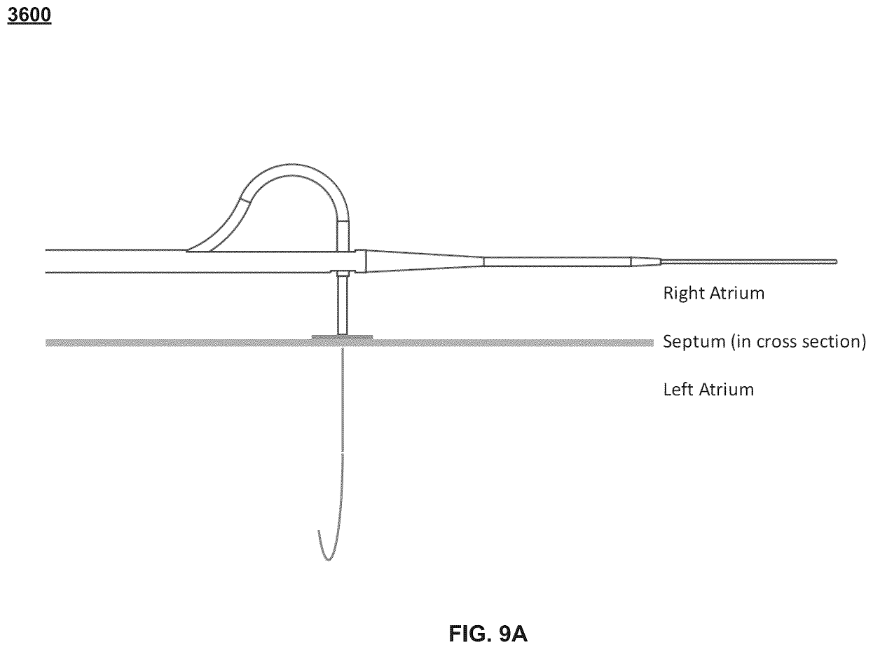

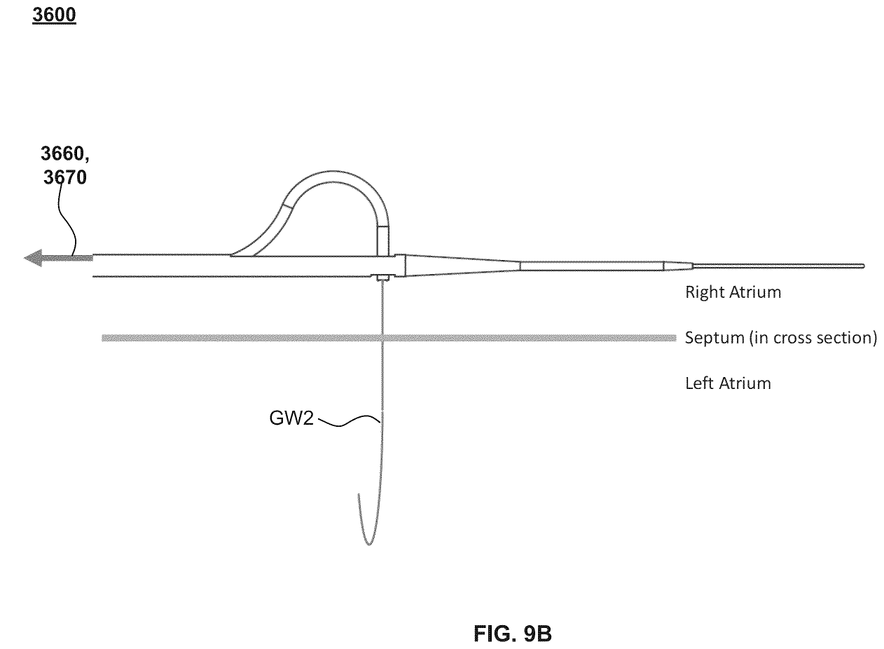

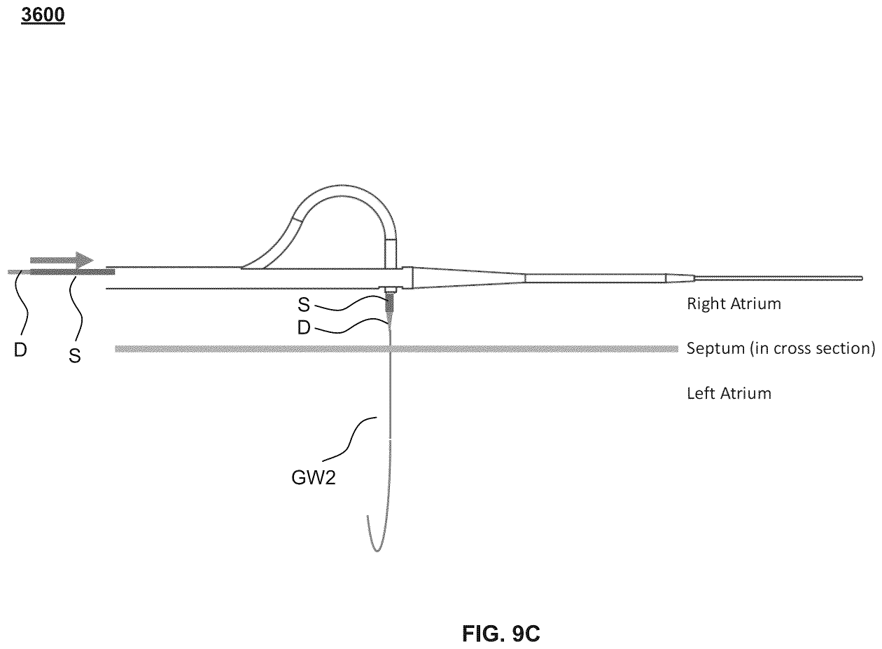

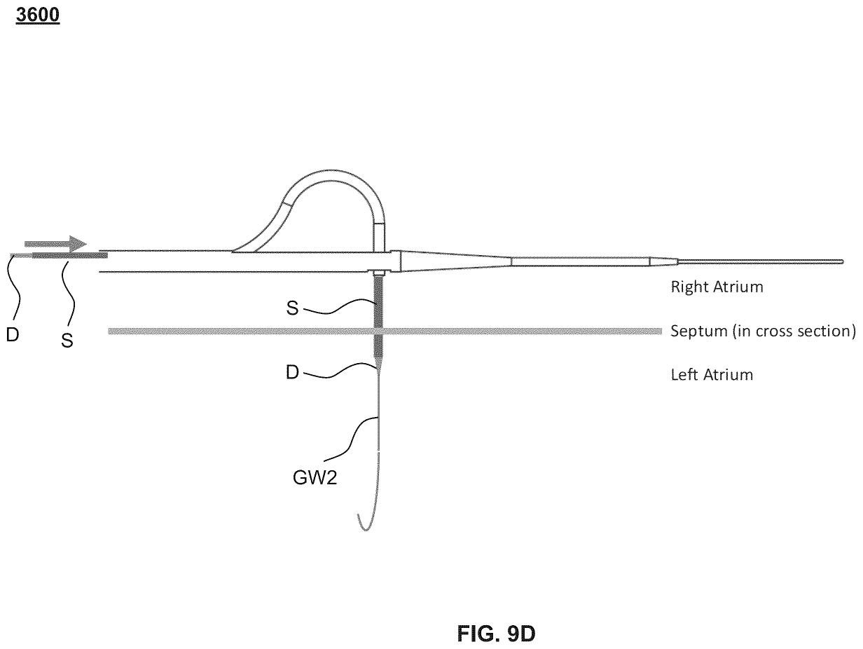

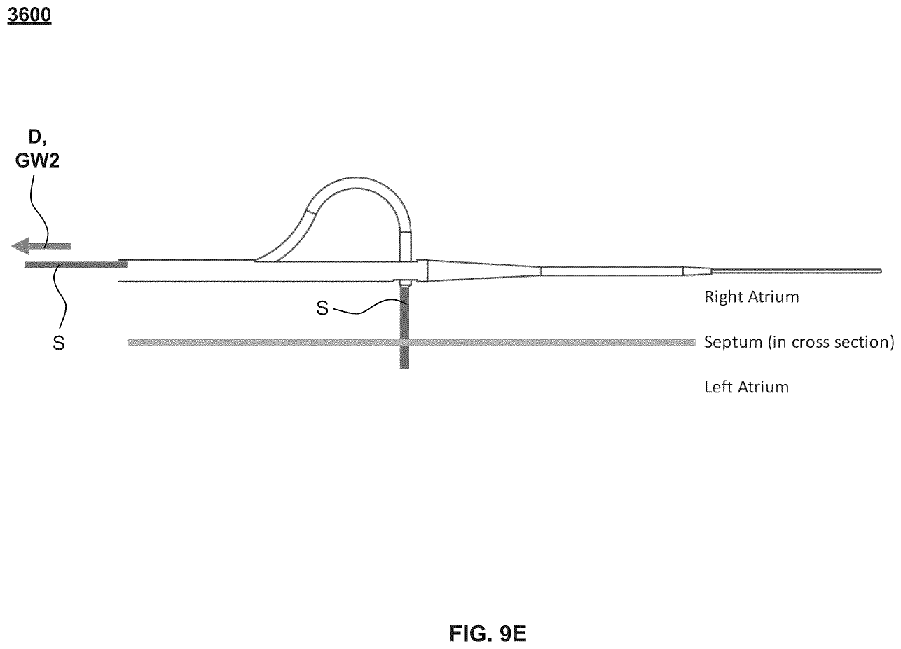

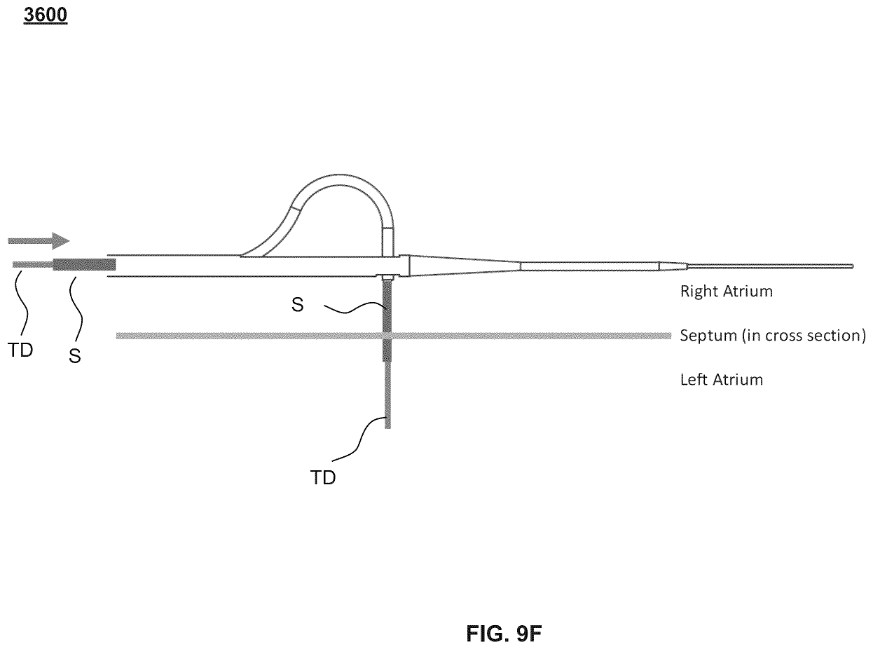

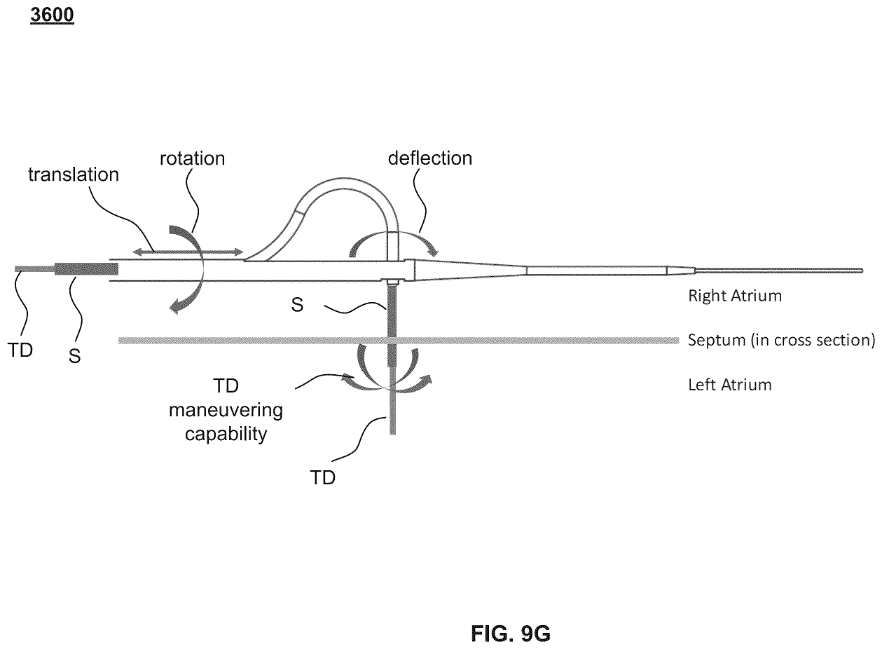

A sequence of operations by which septum puncture device 3600 can be used to assist in delivery of a therapeutic device to the LA, and to assist in maneuvering the therapeutic device in the LA is illustrated in FIGS. 7A to 7G and shown in the flow chart of FIG. 8 as process 200′. As shown in FIG. 7B, septum puncture device 3600 has been used to puncture the septum and deliver a guidewire GW2 into the LA. The sequence of operations leading up to this point are not described here, but may be those described in detail above. Thus, the process in the flow chart of FIG. 8 is enumerated 200′, but starts with a step 220′ that is the same as step 200 from FIG. 3, and omits step 222 from FIG. 3. As shown in FIG. 7C, and in step 224′ in FIG. 9, the septum penetrator 3670 can be retracted into side catheter 3660, e.g. for safety, leaving the guidewire GW2 in the LA. As shown in FIG. 7D, and in step 226′ in FIG. 8, the side catheter 3660, end effector 3662, and septum penetrator 3670 are withdrawn proximally through side catheter guide 3630, and removed from septum puncture device 3600. As shown in FIG. 7E, and in step 228′ in FIG. 8, a therapeutic device TD can then be inserted into septum penetrator device 3600, and delivered distally through the lumen of side catheter guide 3630, over guidewire GW2 to aid in crossing the septum, until its distal end is disposed in the LA. As shown in FIG. 7F, and in step 230′ in FIG. 8, guidewire GW2 can then be withdrawn proximally through the guidewire lumen of therapeutic device TD, and removed from septum puncture device 3600. As shown in FIG. 7G, and in step 232′ in FIG. 8, the therapeutic device TD (e.g. the distal end or a more proximal portion thereof) can then be maneuvered within the LA using its own maneuvering capabilities. In addition, the therapeutic device TD can be maneuvered through additional degrees of freedom by maneuvering septum puncture device 3600 using its capabilities, e.g. by rotation and translation of main shaft 3620 and through deflection of side catheter guide 3630 (which provide a rotational degree of freedom transverse to the axis of main shaft 3620). After therapeutic device TD has been used for the balance of the clinical procedure for which it has been deployed, therapeutic device TD can be withdrawn proximally through the lumen of side catheter guide 3630 and removed from septum puncture device 3600, as shown in step 234′ in FIG. 8. Optionally, as shown in step 236′ in FIG. 8, a second therapeutic device could be delivered through septum puncture device, e.g. by delivering a second guidewire through the first therapeutic device TD before withdrawing it, and delivering the second therapeutic device over the second guidewire. Once the clinical procedure has been completed, the septum puncture device can be removed from the patient, as described in detail above and identified in step 238′ in FIG. 8.

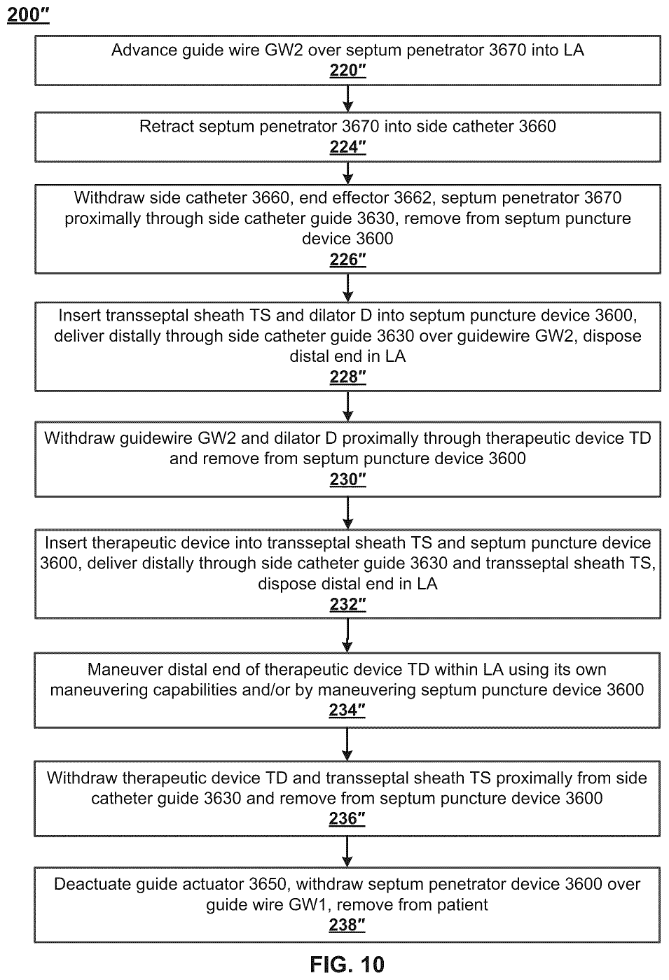

A similar sequence of operations by which septum puncture device 3600 can be used to assist in delivery of a therapeutic device to the LA, and to assist in maneuvering the therapeutic device in the LA is illustrated in FIGS. 9A to 9G, and shown in the flow chart of FIG. 10 as process 200″. This sequence of operations differs from that shown in FIGS. 7A to 7G in that a septum puncture device 3600 can be used to assist in delivery of a transseptal sheath to the LA, and the therapeutic device TD can then be delivered through the transseptal sheath. The first two steps, shown in FIGS. 9A and 9B, and in steps 224″ and 226″ are the same as those shown in FIGS. 7C and 7D, i.e. the septum penetrator 3670 can be retracted into side catheter 3660, e.g. for safety (FIG. 9A, step 224″) and the side catheter 3660, end effector 3662, and septum penetrator 3670 are withdrawn proximally through side catheter guide 3630, and removed from septum puncture device 3600 (FIG. 9B, step 226″). As shown in FIGS. 9C and 9D, and step 228″ in FIG. 10, a transseptal sheath TS and dilator D (e.g. a Mullins type sheath/dilator set) can then be inserted into septum puncture device 3600, and delivered distally through the lumen of side catheter guide 3630, over guidewire GW2 to aid in crossing the septum, until their distal ends are disposed in the LA. As shown in FIG. 9E, and step 230″ in FIG. 10, dilator D and guidewire GW2 can then be withdrawn proximally through the lumen of transseptal sheath TS, and removed from septum puncture device 3600. As shown in FIG. 9F, and in step 232″ in FIG. 10, therapeutic device TD can then be inserted into transseptal sheath TS and septum puncture device 3600, and delivered distally through the lumen of transseptal sheath TS, until its distal end is disposed in the LA. As shown in FIG. 9G, and step 234″ in FIG. 10, the therapeutic device TD (e.g. the distal end or a more proximal portion thereof) can then be maneuvered within the LA using its own maneuvering capabilities, in the same manner as described above with reference to FIG. 7G. Subsequently, as shown in step 236″ in FIG. 10, the therapeutic device TD and transseptal sheath TS can be withdrawn proximally from side catheter guide 3630 and removed from septum puncture device 3600. Finally, in step 238″, guide actuator 3630 can be deactuated and septum puncture device 3600 can be withdrawn over guidewire GW1 and removed from the patient.

Detailed embodiments of the present disclosure have been disclosed herein or purposes of describing and illustrating claimed structures and methods that can be embodied in various forms, and are not intended to be exhaustive in any way, or limited to the disclosed embodiments. Many modifications and variations will be apparent without departing from the scope of the disclosed embodiments. The terminology used herein was chosen to best explain the principles of the one or more embodiments, practical applications, or technical improvements over current technologies, or to enable understanding of the embodiments disclosed herein. As described, details of well-known features and techniques can be omitted to avoid unnecessarily obscuring the embodiments of the present disclosure.

References in the specification to “one embodiment,” “an embodiment,” “an example embodiment,” or the like, indicate that the embodiment described can include one or more particular features, structures, or characteristics, but it shall be understood that such particular features, structures, or characteristics may or may not be common to each and every disclosed embodiment disclosed herein. Moreover, such phrases do not necessarily refer to any one particular embodiment per se. As such, when one or more particular features, structures, or characteristics is described in connection with an embodiment, it is submitted that it is within the knowledge of those skilled in the art to affect such one or more features, structures, or characteristics in connection with other embodiments, where applicable, whether or not explicitly described.

Parameters, dimensions, materials, and configurations described herein are meant to be examples and that the actual parameters, dimensions, materials, and/or configurations will depend upon the specific application or applications for which the inventive teachings is/are used. It is, therefore, to be understood that the foregoing embodiments are presented by way of example only and that, within the scope of the appended claims and equivalents thereto; and that embodiments can be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

As you herein, the phrase “and/or” should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” phrase, whether related or unrelated to those elements specifically identified. Thus, as a non-limiting example, a reference to “A and/or B”, when used in conjunction with open-ended language such as “comprising” or “including” can refer, in one embodiment, to A only (optionally including elements other than B); in another embodiment, to B only (optionally including elements other than A); in yet another embodiment, to both A and B (optionally including other elements); etc.

As used herein, the term, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of.” “Consisting essentially of,” when used in the claims, shall have its ordinary meaning as used in the field of patent law.

As used herein, the terms “about” and/or “approximately” when used in conjunction with values and/or ranges generally refer to those values and/or ranges near to a recited value and/or range. In some instances, the terms “about” and “approximately” may mean within ±10% of the recited value. For example, in some instances, “approximately a diameter of an instrument” may mean within ±10% of the diameter of the instrument. The terms “about” and “approximately” may be used interchangeably. Similarly, the term “substantially” when used in conjunction with physical and/or geometric feature(s), structure(s), characteristic(s), relationship(s), etc. is intended to convey that the feature(s), structure(s), characteristic(s), relationship(s), etc. so defined is/are nominally the feature(s), structure(s), characteristic(s), relationship(s), etc. As one example, a first quantity that is described as being “substantially equal” to a second quantity is intended to convey that, although equality may be desirable, some variance can occur. Such variance can result from manufacturing tolerances, limitations, approximations, and/or other practical considerations. Thus, the term “substantially.”

While various embodiments have been described above, it should be understood that they have been presented by way of example only, and not limitation. Where schematics and/or embodiments described above indicate certain components arranged in certain orientations or positions, the arrangement of components may be modified. While the embodiments have been particularly shown and described, it will be understood that various changes in form and details may be made. Although various embodiments have been described as having particular features and/or combinations of components, other embodiments are possible having a combination of any features and/or components from any of embodiments described herein.

The specific configurations of the various components can also be varied. For example, the size and specific shape of the various components can be different from the embodiments shown, while still providing the functions as described herein. More specifically, the size and shape of the various components can be specifically selected for a desired or intended usage. Thus, it should be understood that the size, shape, and/or arrangement of the embodiments and/or components thereof can be adapted for a given use unless the context explicitly states otherwise.

Where methods and/or events described above indicate certain events and/or procedures occurring in certain order, the ordering of certain events and/or procedures may be modified. Additionally, certain events and/or procedures may be performed concurrently in a parallel process when possible, as well as performed sequentially as described above.

1. An apparatus, comprising:
a shaft;
a guide coupled and angularly deflectable relative to the shaft via a guide coupler, the guide configured to be transitioned between a delivery configuration and a deployed configuration in which a distal end of the guide points away from a centerline of the shaft when transitioned from its delivery configuration to its deployed configuration, the guide defining a lumen;
an elongate member removably and slidably disposable within the lumen of the guide and configured to extend distally relative to the distal end of the guide, the elongate member defining a lumen; and
a puncture member slidably disposable within the lumen of the elongate member and configured to extend distally relative to a distal end of the elongate member, the puncture member configured to puncture tissue of a patient,
the guide further being configured to have the elongate member and the puncture member removed from the lumen of the guide and to receive in the lumen of the guide a therapeutic device, and to be angularly deflected relative to the shaft while the therapeutic device is disposed in the lumen of the guide.

2. The apparatus of claim 1, further comprising:
an end effector disposed about the elongate member, having a cross-sectional area greater than a cross-sectional area of the elongate member, and removably and slidable disposable within the lumen of the guide, the guide further being configured to have the end effector removed from the lumen of the guide with the elongate member and the puncture member.

3. The apparatus of claim 2, wherein
the guide is configured to be transitioned between its delivery configuration and its deployed configuration in response to relative movement between (1) a portion of the guide disposed proximal to the guide coupler and (2) the shaft.

4. The apparatus of claim 1, wherein:
the puncture member defines a lumen configured to slidably receive a guide wire.

5. The apparatus of claim 1, wherein:
the guide coupler allows rotational movement of the guide relative to the shaft and limits relative linear movement between the guide coupler and the guide.

6. The apparatus of claim 1, wherein:
the guide coupler is a hinge.

7. The apparatus of claim 1, wherein:
the guide coupler is spaced proximally from a distal end of the shaft.

8. The apparatus of claim 1, wherein the guide is further configured to receive in the lumen of the guide a sheath, and the therapeutic device within the sheath, and to be angularly deflected relative to the shaft while the sheath and the therapeutic device is disposed in the lumen of the guide.

9. The apparatus of claim 1, wherein:
the distal end of the guide is configured to be angularly deflected when transitioned from its delivery configuration to its deployed configuration such that a central axis defined by the distal end of the guide is about perpendicular relative to a central axis of the shaft.

10. A system, comprising:
a catheter having:
a shaft; and
a guide coupled and angularly deflectable relative to the shaft via a guide coupler, the guide configured to be transitioned between a delivery configuration and a deployed configuration in which a distal end of the guide points away from a centerline of the shaft when transitioned from its delivery configuration to its deployed configuration, the guide defining a lumen; and
a therapeutic device disposed in the lumen of the guide, with a distal end of the therapeutic device extending from the distal end of the guide, the position of the therapeutic device being controllable by one or more of angular deflection of the guide, translation of the shaft, and rotation of the shaft.

11. The system of claim 10, further comprising:
a handle operably coupled to the shaft and the guide, the shaft and the guide extending distally from the handle, the guide being coupled to the shaft via a guide coupler,
a length of the guide disposed between a distal end of the handle and the guide coupler increases in response to the guide being transitioned from its delivery configuration to its deployed configuration.

12. The system of claim 11, wherein:
the guide coupler allows rotational movement of the guide relative to the shaft but limits relative linear movement between the guide and the guide coupler.

13. The system of claim 10, wherein the guide is coupled to the shaft via a guide coupler, the guide coupler being a hinge.

14. The system of claim 13, wherein the guide coupler is spaced proximally from a distal end of the shaft.

15. The system of claim 10, further comprising a sheath disposed in the lumen of
the guide with a distal end of the sheath extending from the distal end of the guide, the therapeutic device disposed in a lumen of the sheath, the distal end of the therapeutic device extending from the distal end of the sheath.

16. The system of claim 10, wherein:
the distal end of the guide is configured to be deflected when transitioned from its delivery configuration to its deployed configuration such that a central axis defined by the distal end of the guide is about perpendicular relative to a central axis of the shaft.

17. A method, comprising:
inserting a shaft having a guide attached thereto via a guide coupler into a heart of a patient such that a distal end of the guide is disposed in the right atrium of the heart;
with the distal end of the guide disposed in the right atrium, deflecting the distal end of the guide about the guide coupler such that the distal end of the guide points towards a septum of the heart;
extending an elongate member that is disposed within the guide distally from the guide and towards the septum;
extending a septum penetrator that is slidably disposed within the elongate member distally from the elongate member such that the septum penetrator pierces the septum at a target puncture site to form a puncture and enters a left atrium of the heart;
withdrawing the elongate member and septum penetrator proximally through the guide and removing them from the guide;
inserting a therapeutic device into the guide; and
delivering the therapeutic device distally through the guide to dispose a distal end of the therapeutic device in the left atrium.

18. The method of claim 17, further comprising maneuvering the therapeutic device within the left atrium by at least one of the translating the shaft, rotating the shaft, or deflecting the guide.

19. The method of claim 18, wherein the therapeutic device has maneuvering capabilities, and the maneuvering the therapeutic device includes using the maneuvering capabilities of the therapeutic device.

20. The method of claim 17, further comprising:
withdrawing the therapeutic device proximally through the guide and removing the therapeutic device from the guide; and
withdrawing the shaft from the heart of the patient.

21. The method claim 17,
further comprising, with the septum penetrator disposed in the left atrium, extending distally a guide wire from within a lumen defined by the septum penetrator from the septum penetrator and into the left atrium, and
wherein the delivering the therapeutic device includes delivering the therapeutic device over the guidewire.

22. The method of claim 17, further comprising, before the inserting the therapeutic device into the guide:
inserting into the guide a sheath and a dilator disposed within the sheath;
delivering the dilator and the sheath distally through the guide to dispose a distal end of the dilator in the left atrium, thereby dilating the puncture with the dilator, and to dispose a distal end of the sheath in the left atrium; and
withdrawing the dilator proximally through the guide and removing the dilator from the guide, and
wherein the inserting the therapeutic device into the guide includes inserting the therapeutic device into the sheath, and the delivering the therapeutic device distally through the guide includes delivering the therapeutic device distally through the guide.

23. The method of claim 22, further comprising:
with the septum penetrator disposed in the left atrium, extending distally a guide wire from within a lumen defined by the septum penetrator from the septum penetrator and into the left atrium, and wherein the delivering the dilator and sheath includes delivering the dilator over the guidewire; and
before the inserting the therapeutic device into the guide, withdrawing the guidewire proximally through the guide and removing the guidewire from the guide.

24. The method of claim 17, further comprising:
after the extending the septum penetrator, verifying that a distal end portion of the septum penetrator is disposed within the left atrium of a heart.

25. The method of claim 17, wherein the verifying includes at least one of (1) measuring a pressure representative of the left atrium from outside the patient and via the lumen defined by the septum penetrator, or (2) delivering a fluid to the left atrium via the lumen defined by the septum penetrator.
